(19) United States
(12) Patent Application Publication
(10) Pub. No.: US 2016/0022199 A1
(43) Pub. Date: Jan. 28, 2016

(54) PHYSICAL CONDITION PRESENTATION APPARATUS, PHYSICAL CONDITION PRESENTATION SYSTEM, PRESENTED INFORMATION OUTPUT METHOD, AND PRESENTED INFORMATION OUTPUT PROGRAM

(71) Applicant: Seiko Epson Corporation, Tokyo (JP)

(72) Inventors: Jun TAKEUCHI, Tama-shi (JP); Junko HASEGAWA, Koganei-shi (JP)

(21) Appl. No.: 14/801,741

(22) Filed: Jul. 16, 2015

(30) Foreign Application Priority Data
Jul. 22, 2014 (JP) 2014-148963

(51) Int. Cl. A61B 5/00; A61B 5/0205; A61B 5/22

(52) U.S. Cl. CPC A61B 5/486; A61B 5/4866; A61B 5/222; A61B 5/0205; A61B 5/7296; A61B 5/7282; A61B 5/743; A61B 5/0002

(57) ABSTRACT

A physical condition presentation apparatus calculates a point corresponding to an item based on a calculation rule set for the item classified to a behavior executed by a user and interlocked to an index indicating a physical condition of the user and the behavior executed within a predetermined period by the user, and presents presented information including at least one of the calculated point and evaluation of the physical condition of the user according to the point.

This application claims priority to Japanese Patent Application No. 2014-148963, filed Jul. 22, 2014, the entirety of which is hereby incorporated by reference.

### BACKGROUND

1. Technical Field

The present invention relates to a physical condition presentation apparatus, a physical condition presentation system, a presented information output method, and a presented information output program.

2. Related Art

In the related art, exercise support systems prompting users to do exercise appropriate for prevention, improvement, and remedy of lifestyle habit diseases such as diabetes have been known (for example, see JP-A-2012-105756).

The exercise support system disclosed in JP-A-2012-105756 presents a movement path from a current location to a destination location based on blood-sugar level information (including a blood-sugar level of a user and a change tendency of the blood-sugar level), a behavior history (behavior content of the user such as diet content and exercise content), and target consumed calories when the user moves from the current location to the destination location. Thus, when the user moves to a destination along the movement path, the user can be guided to the destination until a requisite arrival time while the user consumes the target consumed calories and the user also does exercise of an appropriate exercise amount and exercise content.

However, respective guidance of various behaviors (for example, diet, exercise, and life habit) included in the daily life of a user is generally executed separately in association with the prevention, improvement, and remedy for the lifestyle habit diseases such as diabetes and obesity. For this reason, not only may a physical condition not be improved in connection with a situation of implementation of such a behavior, but it is also difficult for the user to comprehend how the value of an index indicating a physical condition of the user (for example, hemoglobin A1c (HbA1c) which is an index indicating a diabetes state) specifically changes when the user performs the behavior. Further, there is a problem that it is difficult to maintain motivation.

### SUMMARY

An advantage of some aspects of the invention is that it provides a physical condition presentation apparatus, a physical condition presentation system, a presented information output method, and a presented information output program enabling a physical condition of a user to be easily comprehended.

A physical condition presentation apparatus according to a first aspect of the invention is configured: to calculate a point corresponding to an item based on a calculation rule set for the item classified according to a behavior executed by a user and related to an index indicating a physical condition of the user and the behavior executed within a predetermined period by the user; and to present at least one of the point and evaluation of the physical condition according to the point.

As at least one item related to the index indicating the physical condition of the user, for example, an item for suppressing an increase in the blood-sugar level and an increase in the weight can be exemplified when the index indicating the physical condition is a blood-sugar level control index such as the foregoing HbA1c or glico albumin or Body Mass Index (BMI). As the calculation rule set for the item, for example, a calculation rule can be exemplified in which when a behavior (for example, a behavior for reducing the blood-sugar level or the weight) for improving the physical condition is performed, one of addition and subtraction of the points set in advance in the item included in the behavior is executed; and when a behavior for degrading the physical condition is performed, the other of the addition and the subtraction of the points set in advance is executed.

According to the first aspect of the invention, points based on the calculation rule are calculated for the item classified to a behavior (a behavior included in a daily life) performed by the user within a predetermined period. For example, when intake calories of the user are lower than target calories, points based on the calculation rule set for an item of the intake calories are calculated. For example, when the user exercises longer than a target exercise time, points based on the calculation rule set for an item of the exercise time are calculated. Then, presented information including at least one of the points and evaluation of the physical condition according to the points is presented.

Accordingly, the content of the behavior related to the index indicating the physical condition of the user using the physical condition presentation apparatus can be digitized. In other words, for example, the index is an index of a blood-sugar level or an index of weight. When a behavior for reducing the blood-sugar level or the weight is performed, the value of the index changed by the behavior and the calculated points can be connected with each other. Therefore, when the points are presented, the user can simply comprehend whether to perform a behavior for improving the physical condition by recognizing the presented points. When the evaluation is presented, the user can more specifically comprehend the evaluation of the physical condition obtained by performing the behavior.

For example, when the calculation rule is adopted in which the points are added as the user performs the behavior of a direction for improving the physical condition, the increase in the points can lead to increase motivation for the behavior for improving the physical condition.

In the first aspect of the invention, it is preferable that the predetermined period is a period longer than one day. The point is preferably calculated per day and is integrated during the predetermined period.

According to the first aspect of the invention described above, since the points are calculated per day, erroneous calculation can be prevented more than when the points according to a behavior performed within a predetermined period are calculated at a once. Further, by presenting the points calculated per day and the evaluation, the user can look back on the daily behavior in consideration of the improvement in the physical condition.

Since the physical condition is changed in an accumulation manner of the daily behavior, the points can be calculated and evaluated according to the change in the actual physical condition of the user by presenting at least one of the points integrated in the predetermined period and the evaluation based on the points.

In the first aspect of the invention, it is preferable that the index indicating the physical condition is an index indicating a health state related to a lifestyle habit disease.

A ratio in which a health state associated with lifestyle habit diseases depends on the behavior content of the daily life is high.

Therefore, according to the first aspect of the invention described above, it is possible to digitize the behavior content of the user for an item included in the behavior related to the lifestyle habit diseases among daily behaviors of the user. Thus, it is possible to calculate and evaluate the points according to the health state related to the lifestyle habit diseases of the user.

In the first aspect of the invention, it is preferable that the health state related to the lifestyle habit disease is a diabetes state, and an index indicating the diabetes state is one of hemoglobin A1c and glico albumin in blood.

According to the first aspect of the invention described above, by adopting a calculation rule set in items contributing to prevention, improvement, and remedy for diabetes among the items classified to the behaviors of the user, it is possible to calculate the points according to the diabetes state as the calculation rule. Thus, even when the user merely confirms the presented integrated value or evaluation, the user can comprehend the diabetes state.

Here, the value of each of hemoglobin A1c and glico albumin is generally used as an index indicating a diabetes state. A measurement method and a determination method were also established.

Therefore, according to the first aspect of the invention described above, it is possible to match the points and the diabetes state relatively easily.

In the first aspect of the invention, it is preferable that, when the index indicating the diabetes state is the hemoglobin A1c, the predetermined period is a period equal to or greater than past 60 days and equal to or less than past 90 days from a current date.

Based on the value of hemoglobin A1c, the average blood-sugar level up to blood collection from two months before of the blood collection can be determined.

Therefore, a period (that is, the predetermined period) in which the points are calculated can match a determination period of the average blood-sugar level based on the value of hemoglobin A1c. Furthermore, the change in the points can match the change in the blood-sugar level. Thus, since the points are presented and the user can confirm the change in the points in the predetermined period, the user can comprehend the condition of diabetes more easily.

In the first aspect of the invention, it is preferable that, when the index indicating the diabetes state is the glico albumin, the predetermined period is a period equal to or greater than past 1 week and equal to or less than past 2 weeks from a current date.

Here, based on the value of glico albumin, the average blood-sugar level from the last month (particularly, most recently two weeks) of blood collection to blood collection can be determined.

Therefore, according to the first aspect of the invention described above, as described above, the period (that is, the predetermined period) in which the points are calculated can match a determination period of the average blood-sugar level based on the value of glico albumin. Furthermore, the change in the points can match the change in the blood-sugar level. Thus, since the points are presented and the user can confirm the change in the points in the predetermined period, the user can comprehend the condition of diabetes more easily.

In the first aspect of the invention, it is preferable that the behavior includes at least diet, exercise, and life habit.

Here, examples of prevention, improvement, and remedy for lifestyle habit diseases include dietary therapy and exercise therapy, but a disorder of lifestyle habit is related to progress of lifestyle habit diseases.

Therefore, according to the first aspect of the invention described above, since at least diet, exercise, and lifestyle habit are included in the behaviors for which the points are calculated, it is possible to appropriately calculate the points of the items classified to the behaviors related to the lifestyle habit diseases. Thus, comprehensive evaluation including the diet, the exercise, and the lifestyle habit can be presented to the user rather than separate evaluation of an execution state of the items included in the diet, the exercise, and the lifestyle habit.

In the first aspect of the invention, it is preferable that the behavior includes exercise, behavior information regarding the behavior of the user detected by a detection apparatus is acquired and the point in regard to exercise is calculated based on the behavior of the user indicated by the acquired behavior information.

Here, it is troublesome for the user to determine whether exercise performed by the user is, for example, exercise of intensity and an exercise amount sufficient for improvement of the lifestyle habit disease.

On the other hand, according to the first aspect of the invention described above, the points in regard to exercise are calculated based on the behavior of the user indicated by the behavior information acquired from the detection apparatus and the calculation rule. Accordingly, it is not necessary to determine whether the exercise is the intensity and the exercise amount of the exercise performed by the user and such determination can be appropriately executed. Thus, it is possible to appropriately calculate the points of the behavior classified to the exercise.

In the first aspect of the invention, it is preferable that the detection apparatus is at least one of a pulsimeter detecting a pulse rate of the user based on a pulse wave and an active amount meter calculating consumed calories of the user.

According to the first aspect of the invention described above, since the detection apparatus is at least one of the pulsimeter and the active amount meter, it is possible to more appropriately comprehend the exercise content (for example, an exercise amount and exercise intensity) of the user based on one of the pulse rate and the consumed calories. Therefore, it is possible to more appropriately calculate the points of the item classified to the exercise.

In the first aspect of the invention, it is preferable that the calculated point is corrected according to a stress state of the user.

Here, the physical condition is changed according to a daily stress state of the user. For example, a blood-sugar level has a tendency to decrease due to improvement or the like of insulin resistance by exercise. However, when the user feels stress, a blood sugar level rarely decreases and rather increases even in a case in which the user performs exercise. Further, physical strength is improved by exercise. However, between when the user feels stress and does not feel stress, an improvement state of physical strength is changed even in the case in which the user performs exercise.

Therefore, according to the first aspect of the invention described above, it is possible to calculate the points more suitable for the physical condition of the user by correcting the points according to the stress state of the user. Thus, the points can be appropriately connected to the value of the index, and thus the appropriate evaluation can be presented to the user.

In the first aspect of the invention, it is preferable that the point is calculated based on an exercise distance of the user.

As described above, the exercise has an effect on improvement in both of a physical state and a mental state. On the other hand, in regard to symptom of lifestyle habit diseases such as obesity or diabetes, although there is a limit, there is a tendency to increase the degree of improvement as an exercise distance or a load at the time of exercise is larger.

Therefore, according to the first aspect of the invention described above, the points are calculated based on the exercise distance of the user. Thus, by presenting the calculated points and the evaluation according to the points, not only is the user prompted to perform exercise but also the points can be appropriately calculated for the performed exercise.

A physical condition presentation system according to a second aspect of the invention includes: a detection unit that detects behavior information regarding a behavior of a user; a calculation unit that calculates a point corresponding to an item based on the behavior information and a calculation rule set for the item classified according to the behavior executed by the user and related to an index indicating a physical condition of the user; and a presentation unit that presents at least one of the calculated point and evaluation of the physical condition according to the point.

In the physical condition presentation system according to the second aspect of the invention, it is possible to obtain the same advantages as the physical condition presentation apparatus according to the first aspect of the invention.

In the second aspect of the invention, it is preferable that the physical condition presentation system further includes a first apparatus that includes the detection unit; and a second apparatus that includes the calculation unit and the presentation unit, and the first apparatus transmits the behavior information detected by the detection unit to the second apparatus.

Here, when the apparatus including the detection unit is mounted on the user and the behavior information is detected, the apparatus is necessarily small-sized and lightweight so that a behavior of the user is not intervened.

On the other hand, according to the second aspect of the invention described above, the first apparatus includes the detection unit and the second apparatus includes the calculation unit and the presentation unit. Thus, the first apparatus can be easily configured to be small-sized and lightweight. Thus, the first apparatus can be configured not to interrupt a behavior of the user.

Since the calculation unit is included in the second apparatus different from the first apparatus. Since the second apparatus is not necessarily mounted on the user, a relatively large processing circuit can be mounted. Therefore, since the processing circuit can realize the functions of the calculation unit. Thus, even when a complicated calculation rule is adopted, the calculation unit can reliably execute a process. Likewise, when the presentation unit is configured as a display unit displaying the presented information, the display unit is included in the second apparatus, and thus the display unit having a relatively large screen can be adopted. Therefore, it is possible to easily confirm the presented information.

In the second aspect of the invention, it is preferable that the physical condition presentation system further includes a first apparatus that includes the detection unit and the presentation unit; and a second apparatus that includes the calculation unit, the first apparatus transmits the behavior information detected by the detection unit to the second apparatus, the second apparatus transmits the presented information based on the received behavior information to the first apparatus, and the first apparatus presents the received presented information.

Here, as described above, when the first apparatus including the detection unit is mounted on the user, the first apparatus includes the presentation unit, and thus the user can confirm the presented information at a timing desired by the user.

Since the second apparatus different from the first apparatus includes the calculation unit, as described above, it is possible to realize the function of the calculation unit in a relatively large processing circuit which can be mounted on the second apparatus. Therefore, even when a complicated calculation rule is adopted, the calculation unit can reliably execute a process.

In the second aspect of the invention, it is preferable that the physical condition presentation system further includes a first apparatus that includes the detection unit and the calculation unit; and a second apparatus that includes the presentation unit, the first apparatus transmits the presented information including at least one of the points calculated based on the detected behavior information and the evaluation of the physical condition according to the point to the second apparatus, and the second apparatus presents the received presented information.

When the presentation unit included in the second apparatus presents the evaluation of the physical situation, the first apparatus may transmit the presented information including the evaluation according to the calculated points to the second apparatus or the second apparatus may execute and present the evaluation according to the received points.

According to the second aspect of the invention described above, when the first apparatus calculates the points and evaluates the physical condition according to the points, the second apparatus can be configured as a display apparatus displaying the presented information and an audio output apparatus outputting the presented information as audio. Accordingly, the second apparatus can be simply configured. Thus, it is possible to simplify the configuration of the physical condition presentation system.

On the other hand, when the first apparatus calculates the points and the second apparatus executes and presents the evaluation of the physical condition according to the received points, the processes can be shared between the first and second apparatuses. Thus, it is possible to prevent a processing load of one of the first and second apparatuses from being increased considerably more than that of the other apparatus.

In the second aspect of the invention, it is preferable that the detection unit includes a first apparatus that includes the calculation unit and the presentation unit and a second apparatus that assists the first apparatus.

Examples of the functions of the second apparatus include a function of supplying information necessary to calculate the points and evaluate the physical condition to the first apparatus and a function of relaying communication between the first apparatus and an apparatus such as a server. An example of the function of relaying the communication between the first apparatus and the apparatus among the functions includes a function of transmitting information (for example, the points) received from the first apparatus to the apparatus.

According to the second aspect of the invention described above, it is possible to expand the function of the first apparatus using the second apparatus. Thus, it is possible to improve versatility of the physical condition presentation system.

A presented information output method according to a third aspect of the invention is executed using a presented information output apparatus that outputs the presented information regarding a physical condition of a user. The method includes: calculating a point corresponding to an item based on a calculation rule set for the item classified according to a behavior executed by the user and related to an index indicating the physical condition of the user and the behavior executed within a predetermined period by the user; and outputting the presented information including at least one of the point and evaluation of the physical condition according to the point.

By executing the presented information output method according to the third aspect of the invention using the presented information output apparatus and presenting the output presented information to the user using the presentation unit such as a display unit, it is possible to obtain the same advantages as the physical condition presentation apparatus according to the first aspect of the invention.

A presented information output program according to a fourth aspect of the invention is executed by a presented information output apparatus that outputs the presented information regarding a physical condition of a user. The program causes the presented information output apparatus: to calculate a point corresponding to an item based on a calculation rule set for the item classified according to a behavior executed by the user and related to an index indicating the physical condition of the user and the behavior executed within a predetermined period by the user; and to output the presented information including at least one of the point and evaluation of the physical condition according to the point.

By causing the presented information output apparatus to execute the presented information output program according to the fourth aspect of the invention and presenting the output presented information to the user using the presentation unit such as a display unit, it is possible to obtain the same advantages as the physical condition presentation apparatus according to the first aspect of the invention.

The presented information output program may be recorded in a computer-readable recording medium. By causing an information processing apparatus (the presented information output apparatus) such as a portable terminal to read the presented information output program from the recording medium and to execute the presented information output program, it is possible to obtain the same advantages as the physical condition presentation apparatus according to the first aspect of the invention. Examples of the recording medium include a magnetic tape, a magnetic disk, an optical disc, a magneto-optical disc, a hard disk drive (HDD), and a semiconductor memory. The presented information output program can be executed in an information processing apparatus by using such a recording medium. Further, the presented information output program may be supplied via a network.

### BRIEF DESCRIPTION OF THE DRAWINGS

The invention will be described with reference to the accompanying drawings, wherein like numbers reference like elements.

FIG. 1 is a schematic diagram illustrating a physical condition presentation system according to a first embodiment of the invention.

FIG. 2 is a block diagram illustrating the configuration of an information processing apparatus according to the first embodiment.

FIG. 3 is a diagram illustrating an example of initial setting information according to the first embodiment.

FIG. 4 is a diagram illustrating an example of target information according to the first embodiment.

FIG. 5 is a diagram illustrating an example of calculation information according to the first embodiment.

FIG. 6 is a block diagram illustrating the configuration of a control unit according to the first embodiment.

FIG. 7 is a diagram illustrating an example of an initial information registration screen according to the first embodiment.

FIG. 8 is a diagram illustrating an example of a target information registration screen according to the first embodiment.

FIG. 9 is a diagram illustrating an example of an exercise result presentation/input screen according to the first embodiment.

FIG. 10 is a diagram illustrating an example of a physical information input screen according to the first embodiment.

FIG. 11 is a diagram illustrating an example of a diet content registration screen according to the first embodiment.

FIG. 12 is a diagram illustrating an example of a simple setting screen according to the first embodiment.

FIG. 13 is a diagram illustrating an example of a sleeping hours registration screen according to the first embodiment.

FIG. 14 is a diagram illustrating an example of a lifestyle content registration screen according to the first embodiment.

FIG. 15 is a diagram illustrating an example of an exercise input screen according to the first embodiment.

FIG. 16 is a diagram illustrating an example of a result presentation screen according to the first embodiment.

FIG. 17 is a diagram illustrating an example of an integrated value presentation screen according to the first embodiment.

FIG. 18 is a diagram illustrating an example of a classified behavior change presentation screen according to the first embodiment.

FIG. 19 is a diagram illustrating an example of a message presentation screen according to the first embodiment.

FIG. 20 is a block diagram illustrating a physical condition presentation system according to a second embodiment of the invention.

FIG. 21 is a block diagram illustrating a detection apparatus included in a physical condition presentation system according to a third embodiment of the invention.

FIG. 22 is a block diagram illustrating an information processing apparatus included in the physical condition presentation system according to the third embodiment of the invention.

FIG. 23 is a block diagram illustrating a physical condition presentation system according to a fourth embodiment of the invention.

### DESCRIPTION OF EXEMPLARY EMBODIMENTS

### First Embodiment

Hereinafter, a first embodiment of the invention will be described with reference to the drawings.

### Schematic Configuration of Physical Condition Presentation System

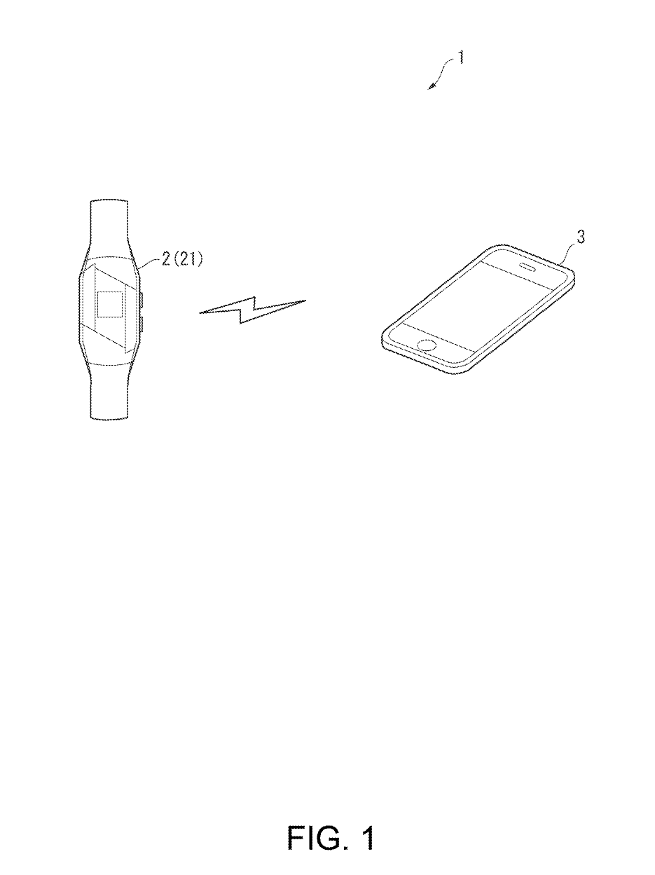

FIG. 1 is a schematic diagram illustrating a physical condition presentation system 1 according to the embodiment.

The physical condition presentation system 1 according to the embodiment includes a detection apparatus 2 and an information processing apparatus 3, as illustrated in FIG. 1. In the physical condition presentation system 1, the information processing apparatus 3 stores initial setting information regarding a physical condition of a user and target information regarding a target value of each of the items classified to behaviors included in a daily life of the user, and also stores calculation information including a calculation rule of a point set for each item. The information processing apparatus 3 calculates a point of each of the items using the calculation information based on an input result input by the user and behavior information of the user detected by the detection apparatus 2, and calculates a sum value of the points per day and an integrated value of the sum values during a retention period which is a predetermined period set in advance. The information processing apparatus 3 presents the user with the calculated sum value and integrated value and evaluation of the physical condition according to the sum value and the integrated value.

Hereinafter, the configuration of the physical condition presentation system 1 will be described.

In the following example, the description will be made assuming that the physical condition presentation system 1 presents health states associated with lifestyle habit diseases as physical conditions of a user, specifically, presents diabetes states.

### Configuration of Detection Apparatus

The detection apparatus 2 is a detection apparatus that is mounted on the user and includes a detection unit 21 detecting information (behavior information) regarding a behavior of the user, and corresponds to a first apparatus according to the invention. The detection apparatus 2 is configured to be able to record and transmit the behavior information.

As the detection apparatus 2, there is exemplified a pulsimeter that detects biometric information such as a pulse rate of the user and an operation information such as acceleration changing with an operation of the user, and records and transmit behavior information including the biometric information and the operation information. Further, as the detection apparatus 2, there is also exemplified an active amount meter that detects the operation information and calculates the number of steps or consumed calories of the user, and records and transmits behavior information including the number of steps or consumed calories of the user.

In the embodiment, the detection unit 21 detects and receives a satellite signal received from a positional information satellite such as a Global Positioning System (GPS) satellite as well as the biometric information and the behavior information, and acquires positional information (that is, positional information indicating a current location of the user on which the detection apparatus 2 is mounted) indicating a current location of the detection apparatus 2. The detection unit 21 acquires the positional information periodically and the detection apparatus 2 transmits the behavior information including the acquired positional information.

The detection apparatus 2 also includes a clocking unit that clocks a current time. The behavior information transmitted by the detection apparatus 2 also includes times at which the biometric information, the behavior information, and the positional information are detected and acquired.

### Configuration of Information Processing Apparatus

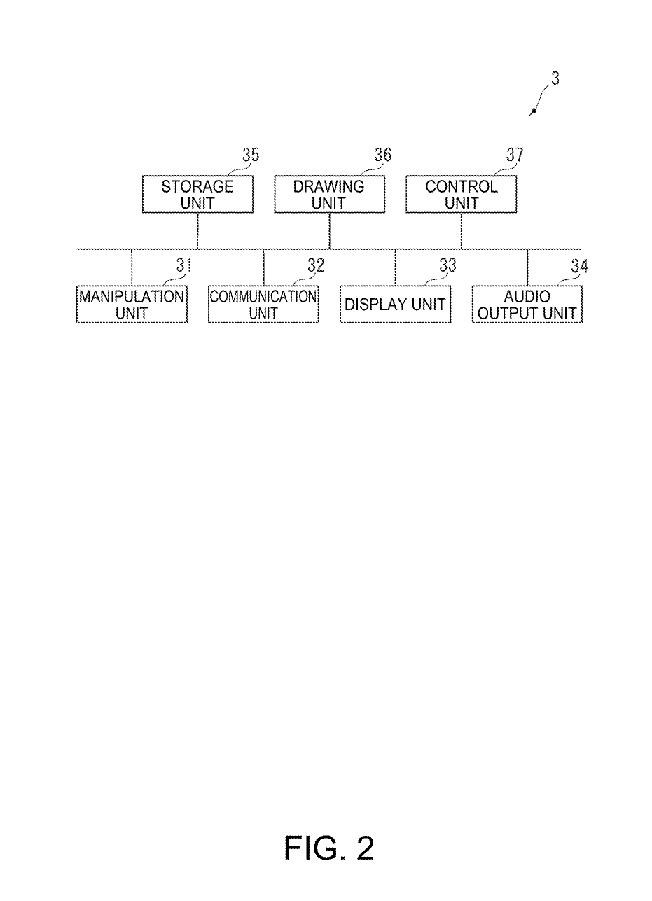

FIG. 2 is a block diagram illustrating the configuration of the information processing apparatus 3.

In the embodiment, the information processing apparatus 3 corresponds to a physical condition presentation apparatus, a second apparatus, and a presented information output apparatus according to the invention and presents, for example, a message based on transition or the like the sum value and the integrated value as well as presenting the points (the sum value and the integrated value) indicating the physical condition of the user and the evaluation of the physical condition according to the points, as described above. The information processing apparatus 3 can be configured by, for example, a smartphone (multi-functional mobile phone), a tablet, or a personal computer (PC).

As illustrated in FIG. 2, the information processing apparatus 3 includes a manipulation unit 31, a communication unit 32, a display unit 33, an audio output unit 34, a storage unit 35, a drawing unit 36, and a control unit 37, which are mutually connected by a bus line.

### Configuration of Manipulation Unit

The manipulation unit 31 receives an input manipulation executed by the user and outputs manipulation information according to the input manipulation to the control unit 37. For example, the manipulation unit 31 can be configured not only by a physical key or a touch panel provided in the casing of the information processing apparatus 3 but also by a keyboard, a pointing device, or the like connected to the information processing apparatus 3 in a wired or wireless manner.

### Configuration of Communication Unit

The communication unit 32 includes a first communication module capable of communicating with an external apparatus such as the detection apparatus 2 and a second communication module capable of communicating with a server (not illustrated) on a network such as the Internet, and communicates with the external apparatus and the server under the control of the control unit 37. When the communication unit 32 communicates with the external apparatus and the server in the same communication scheme, the communication unit 32 may include one of the first and second communication modules or may not include the second communication module when it is not necessary to communicate with the server. When the user can input behavior information regarding the user through an input manipulation on the manipulation unit 31, the connection with the detection apparatus 2 is not requisite. Therefore, in this case, the communication unit 32 may not be provided.

### Configuration of Display Unit

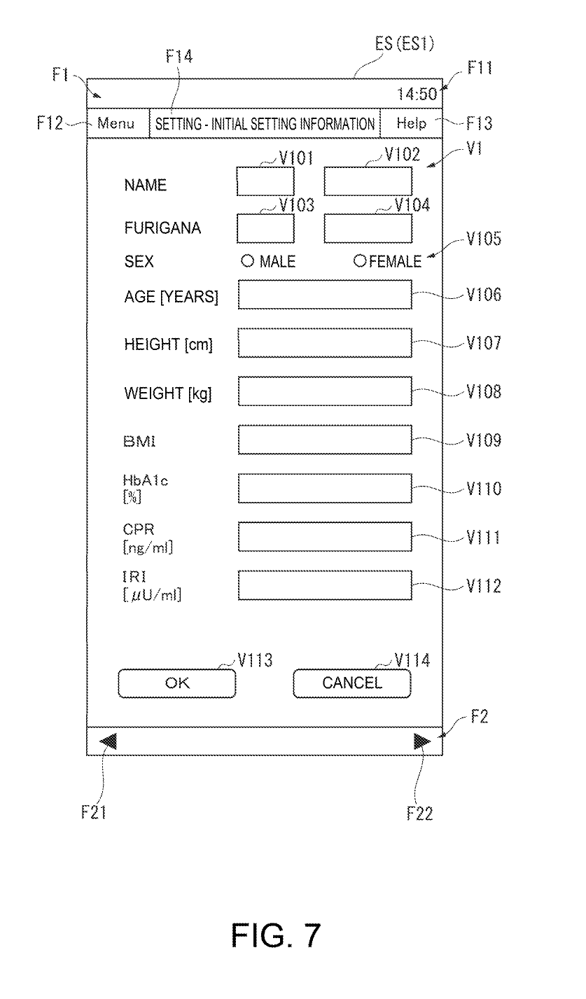

The display unit 33 displays an image drawn by the drawing unit 36. Specifically, the display unit 33 displays execution screens of an operating system (OS) and an application executed by the control unit 37. That is, the display unit 33 displays execution screens ES (see FIGS. 7 to 19) of a physical condition presentation application to be described and functions as a presentation unit that presents various kinds of information to the user. The display unit 33 can be configured by, for example, any of various display panels of liquid crystal, organic electro-luminescence (EL), electrophoresis, and the like.

### Configuration of Audio Output Unit

The audio output unit 34 is configured to include a speaker and outputs audio according to audio information input from the control unit 37. For example, when the control unit 37 executes the physical condition presentation application, the audio output unit 34 outputs audio according to information presented to the user. That is, the audio output unit 34 forms a presentation unit according to the invention, as in the display unit 33.

### Configuration of Storage Unit

The storage unit 35 is configured by a storage device such as a solid state drive (SSD), a hard disk drive (HDD), or a flash memory and stores a program and data necessary for an operation of the information processing apparatus 3. For example, the storage unit 35 stores not only the OS controlling the information processing apparatus 3 as the program but also the physical condition presentation application (including a presented information output program according to the invention) causing the control unit 37 to execute a physical condition presentation process to be described below.

### Initial Setting Information

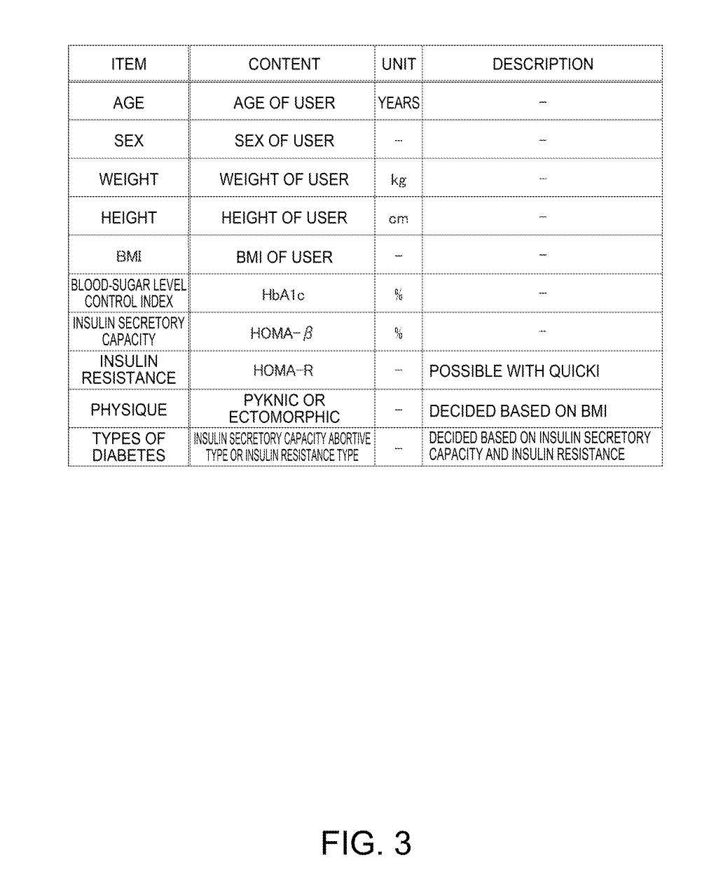

FIG. 3 is a diagram illustrating an example of the initial setting information stored by the storage unit 35.

The storage unit 35 stores the initial setting information to be described below. The initial setting information is, for example, information that is input through an input manipulation of the user on the manipulation unit 31 at the time of the first execution of the physical condition presentation application and is information regarding the physical condition of the user.

As illustrated in FIG. 3, the initial setting information includes, for example, “age,” “sex,” “weight,” “height,” “body mass index (BMI),” “blood-sugar level control index,” “insulin secretory capacity,” “insulin resistance,” “physique,” and “types of diabetes.”

Of the items, the “blood-sugar level control index” is the value of hemoglobin A1c (HbA1c) in the embodiment. The value of HbA1c is a value that is correlated to an average value of blood-sugar levels over 2 months. The “physique” is one of “pyknic” and “ectomorphic” physiques and the “types of diabetes” is one of “insulin secretory capacity abortive type” and “insulin resistance type.” The “insulin secretory capacity” is the value of HOMA-β which is an index of an insulin secretory capacity and the “insulin resistance” is the value of HOMA-R which is an index of insulin resistance. However, the invention is not limited thereto. For example, the “insulin resistance” may be the value of quantitative insulin sensitivity check index (QUICKI) which is an index of the same insulin resistance.

The “age,” “sex,” “weight,” and “blood-sugar control index” are assumed to be requisite items in the embodiment and the other items are assumed to be optional setting items.

Instead of these items or in addition to these items, other items may be included in the initial setting information.

At least one of the foregoing items may be set automatically according to the other items. For example, the BMI can be calculated from height and weight. Therefore, when the “height” and “weight” are included in the initial setting information, the “BMI” may not be input by the user. Likewise, the “physique” can be obtained based on the BMI. Thus, the “types of diabetes” can also be determined from the values of the “insulin secretory capacity” and “insulin resistance.”

### Target Information

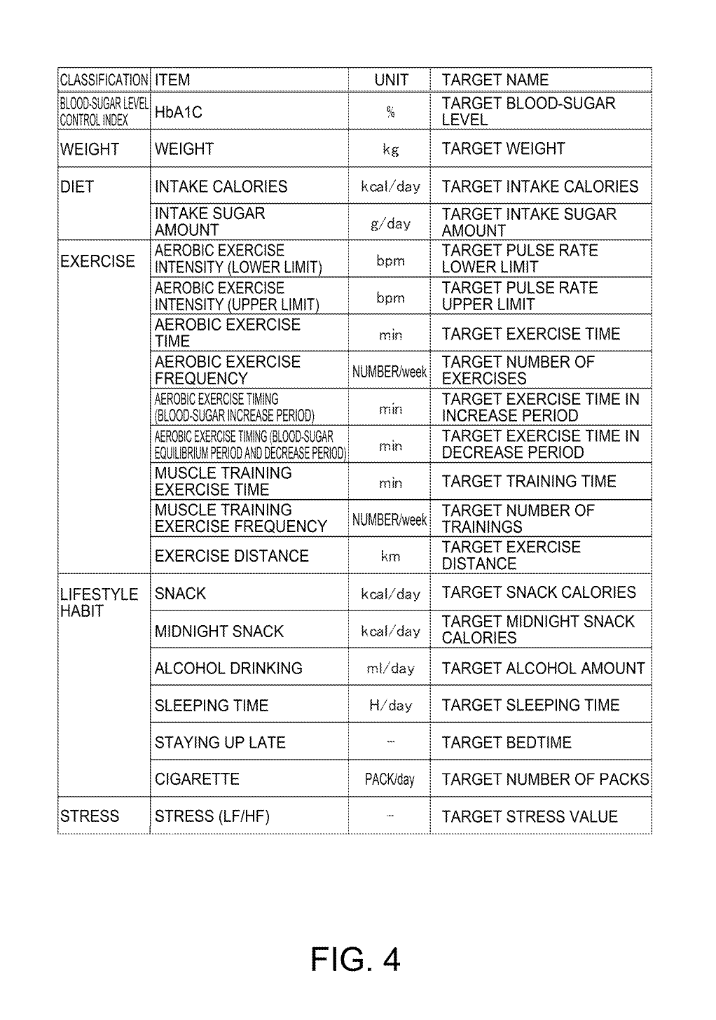

FIG. 4 is a diagram illustrating an example of the target information stored in the storage unit 35.

The storage unit 35 stores target information to be described below.

The target information is information that indicates a target of each of the items classified to behaviors included in a daily life. Specifically, the target information is information that is classified according to the behaviors and indicates a target of each item related to the blood-sugar control index which is an index indicating a physical condition of the user. The target information may be set and input by a user or a medical worker (for example, a physician) or may be set automatically by the information processing apparatus 3 based on the initial setting information.

As illustrated in FIG. 4, the target information includes, for example, a target value (target blood-sugar level) of the “blood-sugar control index” and a target value (target weight) of the “weight” and also includes a target value of each of the items classified in “diet,” “exercise,” and “lifestyle habit” and a target value of “stress.”

The items classified in the “diet” include “intake calories” and “intake sugar amount” in the embodiment.

The “intake calories” is an item in which a target value (target intake calories) of intake calories per day is set and the “intake sugar amount” is an item in which a target value (target sugar amount) of a sugar amount per day is set.

The items classified in the “exercise” include “aerobic exercise intensity (lower limit),” “aerobic exercise intensity (upper limit),” “aerobic exercise time,” “aerobic exercise frequency,” “aerobic exercise timing (blood-sugar increase period),” “aerobic exercise timing (blood-sugar equilibrium period and decrease period),” “muscle training exercise time,” and “muscle training exercise frequency” in the embodiment.

The “aerobic exercise intensity (lower limit)” and “aerobic exercise intensity (upper limit)” are items in which a target value (target pulse rate lower limit) of the lower limit of a pulse rate and a target value (target pulse rate upper limit) of the upper limit of a pulse rate of an aerobic exercise of the user are set.

The “aerobic exercise time” is a target value (target exercise time) of an aerobic exercise time per day and the “aerobic exercise frequency” is an item in which a target value (target number of target exercises) of the number of aerobic exercises per week is set.

The “aerobic exercise timing (blood-sugar increase period)” is an item in which a target value (target exercise time in an increase period) of an aerobic exercise time in a period in which a blood-sugar level increases after diet. For example, 10 minutes are set.

The “aerobic exercise timing (blood-sugar equilibrium period and decrease period)” is an item in which a target value (target exercise time in a decrease period) of an aerobic exercise time set in a period in which a blood-sugar level after diet is in an equilibrium state and a period in which the blood-sugar level continues to decrease. For example, 10 minutes are set.

The “muscle training exercise time” is an item in which a target value (target training time) of a time in which a muscle training is performed per day is set and the “muscle training exercise frequency” is an item in which a target value (target number of trainings) of the number of times the muscle training is performed per week is set. Like the target number of exercises, at the time of counting of the target number of trainings, the number of times the muscle training is performed is counted as one even when the muscle training is performed a plurality of times in a day.

An “exercise distance” is a target value (target exercise distance) of a traveling distance per day. In the embodiment, the target value of the traveling distance at the time of the aerobic exercise is set as the target exercise distance in consideration of a power consumption amount of an apparatus measuring the traveling distance. However, a target value of a total traveling distance for a day may be set.

The items classified in the “lifestyle habit” include “snack,” “midnight snack,” “alcohol drinking,” “sleeping hours,” “staying up late,” and “cigarette” in the embodiment.

The “snack” is an item in which a target value (target snack calories) of the intake calories in snack per day is set and the “midnight snack” is an item in which a target value (target midnight calories) of intake calories 2 hours before going to bet per day is set.

The “alcohol drinking” is an item in which a target value (target alcohol amount) of an intake alcohol amount per day is set. Even when the target alcohol amount is not “0,” drinking alcohol is not recommended.

The “sleeping hours” is an item in which a target value (target sleeping hours) of a sleeping hours per day is set. The “staying up late” is an item in which a target value (target bedtime) of bedtime is set. The “cigarette” is an item in which a target value (target number of packs) of the number of cigarettes per day is set.

The “stress” indicates a balance between sympathetic nerve activity and parasympathetic nerve activity and is an item in which a target value (target stress value) of low frequency (LF)/high frequency (HF) is set as an index (degree of activity of sympathetic nerve) of stress.

HF indicates a sum amount of fluctuation waves which have a period from about 3 seconds to about 4 seconds and uses respiration as a signal source or a power spectrum of its frequency region. On the other hand, LF indicates a sum amount of fluctuation waves which use a change in blood pressure with a period of about 10 seconds called a Mayer wave as a signal source or a power spectrum of its frequency region.

In general, when a value of LF/HF is equal to or less than 2, the stress value is determined to be normal. Therefore, for example, “2” is set as the target stress value.

### Calculation Information

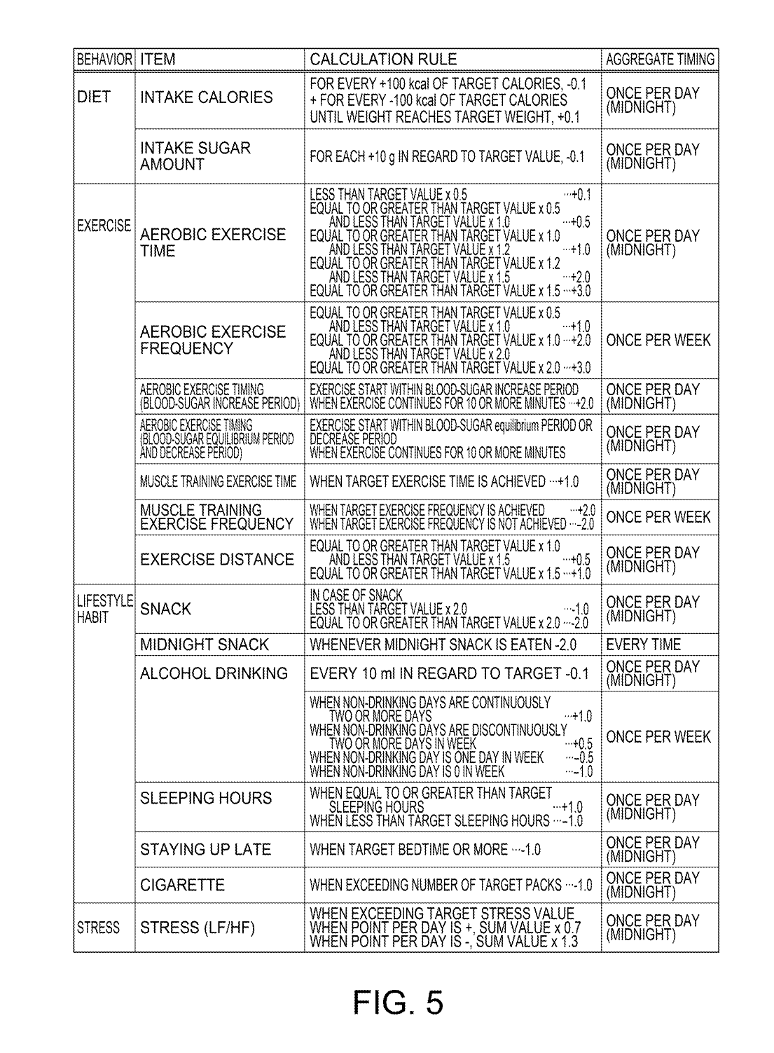

FIG. 5 is a diagram illustrating an example of the calculation information stored in the storage unit 35.

The storage unit 35 stores calculation information to be described below.

The calculation information is information that is used when the behaviors of the user are digitized by the control unit 37 to be described below. As illustrated in FIG. 5, for example, calculation rules are set in the items classified in the “diet,” “exercise,” and “lifestyle habit” included in the daily life of the user and the “stress.”

In the “diet,” the following calculation rules are included in the calculation information.

For the “intake calories,” when intake calories of the user per day exceed the target intake calories, a calculation rule in which −0.1 is set whenever the intake calories exceed the target intake calories by 100 kcal is set. In this calculation rule, when the current weight of the user is greater than the target weight and the intake calories per day are less than the target intake calories, +0.1 is set whenever the intake calories are less than the target intake calories by 100 kcal.

For the “intake sugar amount,” a calculation rule is set in which −0.1 is set whenever the intake sugar amount of the user per day exceeds the target intake sugar amount by 10 g. For normal people, a sugar amount from 100 g to 130 g per day is considered to be necessary. From this viewpoint, in the calculation rule for the intake sugar amount, a point of + is not set when the intake sugar amount per day is less than the target intake sugar amount.

The calculation rules for the “intake calories” and “intake sugar amount” are applied once per day (for example, at a time classified as the night).

For the “exercise,” the following calculation rules are included in the calculation information.

Calculation rules set for the “aerobic exercise time” are applied when an aerobic exercise in a range from the target pulse rate lower limit and the target pulse rate upper limit is performed.

In the calculation rules, when the time of the aerobic exercise performed by the user per day is less than a value obtained by multiplying the target exercise time by 0.5, +0.1 is set. When the time of the aerobic exercise is equal to or greater than a value obtained by multiplying the target exercise time by 0.5 and is less than a value obtained by multiplying the target exercise time by 1.0, +0.5 is set. When the time of the aerobic exercise is equal to or greater than a value obtained by multiplying the target exercise time by 1.0 and is less than a value obtained by multiplying the target exercise time by 1.2, +1.0 is set. When the time of the aerobic exercise is equal to or greater than a value obtained by multiplying the target exercise time by 1.2 and is less than a value obtained by multiplying the target exercise time by 1.5, +2.0 is set. When the time of the aerobic exercise is equal to or greater than a value obtained by multiplying the target exercise time by 1.5, +3.0 is set.

For the “aerobic exercise frequency,” a calculation rule is set in which when the number of aerobic exercises per week is equal to or greater than a value obtained by multiplying the target number of exercises by 0.5 and is less than a value obtained by multiplying the target number of exercises by 1.0, +1.0 is set; when the number of aerobic exercises is equal to or greater than a value obtained by multiplying the target number of exercises by 1.0 and is less than a value obtained by multiplying the target number of exercises by 2.0, +2.0 is set; and when the number of aerobic exercises is equal to or greater than a value obtained by multiplying the target number of exercises by 2.0, +3.0 is set. This calculation rule is applied once per week. At the time of counting of the number of aerobic exercises per week, the number of times the aerobic exercise is performed is counted as one even when the aerobic exercise is performed a plurality of times in a day.

For the “aerobic exercise timing (blood-sugar increase period),” a calculation rule is set in which +2.0 is set when an aerobic exercise starts in a blood-sugar increase period and the aerobic exercise is performed for a time equal to or greater than the target exercise time in the increase period.

Further, for the “aerobic exercise timing (blood-sugar equilibrium period and decrease period),” a calculation rule is set in which +1.0 is set when an aerobic exercise starts in a blood-sugar equilibrium period or decrease period and the aerobic exercise is performed for a time equal to or greater than the target exercise time in the decrease period.

For the “muscle training exercise time,” a calculation rule is set in which +1.0 is set when an exercise time of a muscle training per day is equal to or greater than the target training time.

For the “muscle training exercise frequency,” a calculation rule is set in which +2.0 is set when the number of muscle trainings per week reaches the target number of trainings and −2.0 is set when the number of muscle trainings per week does not reach the target number of trainings. This calculation rule is applied once per week. As in the above-described case, at the time of counting of the number of muscle trainings per week, the number of times the muscle training is performed is counted as one even when the muscle training is performed a plurality of times in a day.

For the “exercise distance,” a calculation rule is set in which +0.5 is set when a traveling distance at the time of an aerobic exercise per day is equal to or greater than the target exercise distance and is less than a value obtained by multiplying the target exercise distance by 1.5, and +1.0 is set when the traveling distance at the time of the aerobic exercise per day is equal to or greater than the value obtained by multiplying the target exercise distance by 1.5.

Of these calculation rules, the calculation rules for the “aerobic exercise time,” “aerobic exercise timing (blood-sugar increase period),” “aerobic exercise timing (blood-sugar equilibrium period and decrease period),” “muscle training exercise time,” and “exercise distance” are applied once per day, as in the calculation rule for the “diet” (for example, at a time classified as the midnight). On the other hand, as described above, the calculation rules for the “aerobic exercise frequency” and “muscle training exercise frequency” are applied once per week.

For the lifestyle habit, the following calculation rules are included in the calculation information.

For the “snack,” a calculation rule is set in which −1.0 is set when snack is eaten in a day (when intake calories by the snack are not 0) and the intake calories per day are less than a value obtained by multiplying the target snack calories by 2.0, and −2.0 is set when the intake calories per day are equal to or greater than the value obtained by multiplying the target snack calories by 2.0.

For the “midnight snack,” a calculation rule is set in which −2.0 is set when diet is eaten 2 hours before going to bed.

For “alcohol drinking,” a calculation rule is set in which −0.1 is set whenever an intake alcohol amount per day exceeds the target alcohol amount by 10 ml.

In this calculation rule, when days (non-drinking days) in which alcohol is not drunk are continuously two or more days in a week, +1.0 is set. When the non-drinking days are discontinuously two or more days in a week, +0.5 is set. When the non-drinking day is one day in the week, −0.5 is set. When there is not non-drinking day in the week, −1 is set. This calculation rule for the alcohol drinking frequency is applied once in a week.

For the “sleeping hours,” a calculation rule is set in which +1.0 is set when the sleeping hours per day is equal to or greater than the target sleeping hours, and −1.0 is set when the sleeping hours per day is less than the target sleeping hours.

For the “staying up late,” a calculation rule is set in which −1.0 is set when the bedtime exceeds the target bedtime.

For the “cigarette,” a calculation rule is set in which −1.0 is set when the number of cigarette packs smoked per day exceeds the target number of packs.

Of the calculation rules, the calculation rules for the “snack,” the intake alcohol amount of “alcohol drinking,” “sleeping hours,” “staying up late,” and “cigarette” are applied once per day (for example, at a time classified as the midnight). On the other hand, as described above, the calculation rule for the alcohol drinking frequency of “alcohol drinking” is applied once per week and the calculation rule for the “midnight snack” is applied whenever the midnight snack is eaten.

For stress, the following calculation rule is included in the calculation information. This calculation rule is applied when the value of LF/HF of the user in a day exceeds the target stress value (when stress is undergone) or when “stress state” is selected by the user on the lifestyle content registration screen ES8 to be described below.

Here, an HF component to which a change in respiration is reflected and an LF component to which a change in blood pressure is reflected are each shown in a relaxed state (a state in which a parasympathetic nerve is activated). On the other hand, the LF component is shown and the HF component decreases in a stress state (a state in which the sympathetic nerve is activated). Thus, in the relaxed state, the value of LF/HF decreases because the HF component relatively increases. In contrast, in the stress state, the value of LF/HF increases because the LF component increases with respect to the HF component.

Thus, by determining whether the value of LF/HF exceeds a predetermined value, it is possible to determine whether the user is in a stress state. The predetermined value is set as the foregoing target stress value.

The calculation rule for the stress is applied when an average value of the values of LF/HF in a day exceeds the target stress value (at the time of the stress state). In the calculation rule for the stress, when a sum value of the points calculated in the calculation rules set in the items of the diet, the exercise, and the lifestyle habit is a + value, a value obtained by multiplying the sum value by 0.7 is set as a sum value after correction. When the sum value of the calculated points is a − value, a value obtained by multiplying the sum value by 1.3 is set as a sum value after correction.

Since the calculation rule for the stress is applied at a timing at which a sum value of the points of the items is calculated, the calculation rule is applied once per day (for example, at a time classified as the midnight).

Thus, a target value and a calculation rule are set for each behavior and each item so that + points are added when a behavior (in the embodiment, a behavior for making diabetes better) for making a health state better is performed, and − points are added when a behavior (in the embodiment, a behavior for making diabetes worse) for making the health state worse is performed.

The invention is not limited thereto. The calculation rule may be set for each behavior and item so that − points are added when a behavior for making a health state better is performed, and + points are added when a behavior for making the health state worse is performed. Further, the points to be added may be appropriately modified.

### Configuration of Drawing Unit

The drawing unit 36 includes a drawing circuit and draws an image to be displayed on the display unit 33 under the control of the control unit 37. For example, the drawing unit 36 draws an execution screen when the control unit 37 executes the physical condition presentation application stored in the storage unit 35. That is, the drawing unit 36 forms a presentation unit according to the invention.

Configuration and operations of various screens included in the execution screen will be described below in detail.

### Configuration of Control Unit

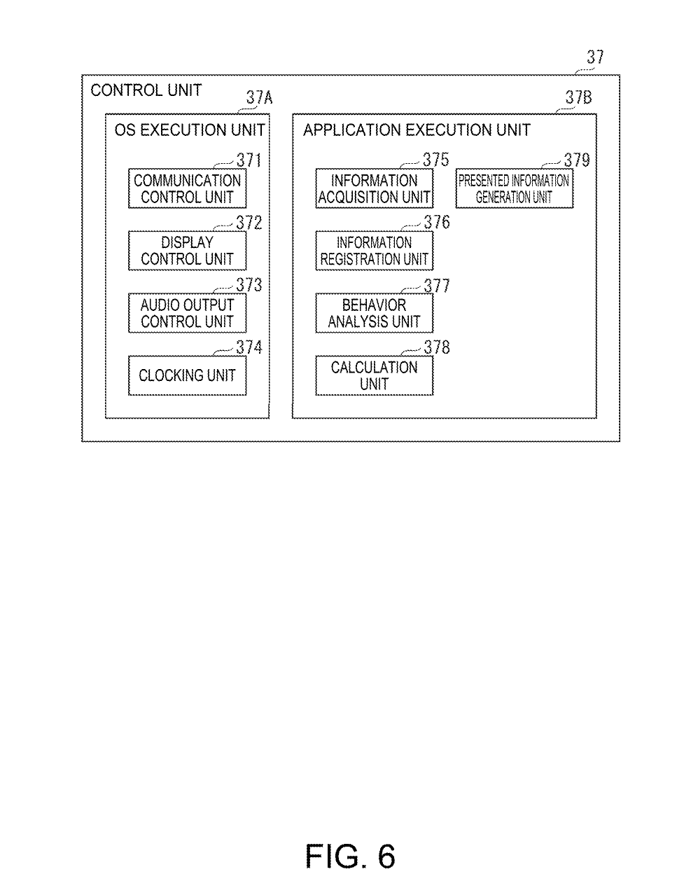

FIG. 6 is a block diagram illustrating the configuration of the control unit 37.

The control unit 37 is configured to include a central processing unit (CPU) and controls an operation of the information processing apparatus 3 by executing a program stored in the storage unit 35. The control unit 37 includes an OS execution unit 37A and an application execution unit 37B.

The OS execution unit 37A is a function unit that executes an OS stored in the storage unit 35 and includes a communication control unit 371, a display control unit 372, an audio output control unit 373, and a clocking unit 374.

The communication control unit 371 controls the communication unit 32 to communicate with the external apparatus or the server.

The display control unit 372 controls the drawing unit 36 such that the drawing unit 36 draws the OS or the execution screen for an application and the display unit 33 displays the execution screen.

The audio output control unit 373 outputs audio information regarding audio output at the time of execution of the OS or the application to the audio output unit 34. That is, the display control unit 372 and the audio output control unit 373 form the presentation unit according to the invention.

The clocking unit 374 clocks a current time.

The application execution unit 37B executes an application instructed by the OS execution unit 37A according to manipulation information input from the manipulation unit 31 among applications stored in the storage unit 35.

The application execution unit 37B includes an information acquisition unit 375, an information registration unit 376, a behavior analysis unit 377, a calculation unit 378, and a presented information generation unit 379 that function by executing the physical condition presentation application stored in the storage unit 35.

The information acquisition unit 375 acquires information input by the user on the execution screen for the physical condition presentation application and also acquires behavior information from the detection apparatus 2 via the communication unit 32.

The information registration unit 376 stores and registers the information acquired by the information acquisition unit 375 in the storage unit 35.

The behavior analysis unit 377 analyzes the behavior information acquired from the detection apparatus 2 and analyzes classification of a behavior performed by the user.

For example, the behavior analysis unit 377 calculates a sum time of an aerobic exercise of a day performed by the user based on the behavior information. Specifically, the behavior analysis unit 377 determines that a behavior of a period equal to or greater than the target pulse rate lower limit and equal to or less than the target pulse rate upper limit and a behavior for which operation information of the period is information including a change corresponding an exercise is an aerobic exercise, and then calculates a sum time of the aerobic exercise of a day as an aerobic exercise time. The behavior analysis unit 377 calculates an exercise distance at the time of the aerobic exercise based on a start time of the aerobic exercise, an end time of the aerobic exercise, and positional information (positional information included in the behavior information) acquired between the start time and the end time of the aerobic exercise.

Further, the behavior analysis unit 377 determines a sleeping onset time and a waking hour of the user based on the behavior information and calculates sleeping hours of the user.

Based on the calculation rule of each of the items, the calculation unit 378 calculates a sum value of the points according to the behavior of the user. Specifically, based on the calculation rules, the calculation unit 378 calculates the sum point of the points according to input information input by the user on the execution screen to be described below and an analysis result (that is, an analysis result of the behavior information) by the behavior analysis unit 377 at a timing (for example, at a time classified as the midnight) described in the calculation rules. The calculation unit 378 corrects the calculated sum value based on the calculation rule for the stress. Further, the calculation unit 378 calculates an integrated value of the sum value during a predetermined period (retention period). The predetermined period will be described below in detail.

The presented information generation unit 379 is a function unit that forms the presentation unit according to the embodiment and generates presentation information (in the embodiment, presentation information including each of a corrected sum value and evaluation of a physical condition of the user according to the sum value) including at least one of the corrected sum value and the evaluation of the physical condition of the user according to the sum value.

The presented information generation unit 379 generates a message according to daily transition of the sum value of the points and the integrated value calculated by the calculation unit 378 and causes the display unit 33 to display the message. Examples of the message include a message regarding the evaluation of the physical condition of the user and a message regarding an execution state of an exercise. The content generated by the presented information generation unit 379 will be described below in detail.

### Configuration of Execution Screen

Here, the display control unit 372 causes the drawing unit 36 to draw execution screens ES (ES1 to ESD) to be described below based on a process result of the application execution unit 37B when the application execution unit 37B executes the physical condition presentation application and causes the display unit 33 to display each execution screen ES.

Hereinafter, the configuration and operation of each execution screen ES will be described.

### Initial Setting Information Registration Screen

FIG. 7 is a diagram illustrating an example of the initial setting information registration screen ES1.

The initial setting information registration screen ES1 is a screen that is included in the execution screen ES and is a screen on which the user information and the initial setting information are input and registered. On the initial setting information registration screen ES1, as illustrated in FIG. 7, fixed display regions F1 and F2 are set in the screen top portion and the screen bottom portion, respectively, and a variable display region V1 is set between the fixed display regions F1 and F2.

A time display region F11 in which a current time clocked by the clocking unit 374 is set is disposed at the upper end of the fixed display region F1 in the screen top portion. A button F12 transitioning to a menu screen (not illustrated) at the time of pressing (inputting) is disposed on the left side of the lower end of the fixed display region F1 and a button F13 transitioning to a help screen (not illustrated) at the time of pressing is disposed on the right side of the lower end of the fixed display region F1. A title F14 indicating content of a screen is disposed in a region between the buttons F12 and F13.

Buttons F21 and F22 are disposed to the left and right of the fixed display region F2 in the screen bottom portion. The buttons F21 and F22 are buttons that are used to transition to a screen.

Entry fields for user information (name of the user) and the initial setting information are provided in the variable display region V1.

Specifically, surname entry fields V101 and V103 for inputting the surname of the user in kanji and hiragana letters of the user and name entry fields V102 and V104 for inputting the name of the user in kanji and hiragana letters among the user information are provided in the variable display region V1.

Entry fields V105 to V112, a registration button V113, and a cancel button V114 for inputting the sex, the age, the height, the weight, the BMI, the blood-sugar level, and a CPR value (a C-peptide value in self-secretion high sensitivity blood at fasting), and an IRI value (an insulin concentration in self-secretion high sensitivity blood at fasting) of the user among the initial setting information are disposed in the variable display region V1. In the sex input field V105 for inputting the sex of the user among these fields, radio buttons for selecting “male” and “female” are provided.

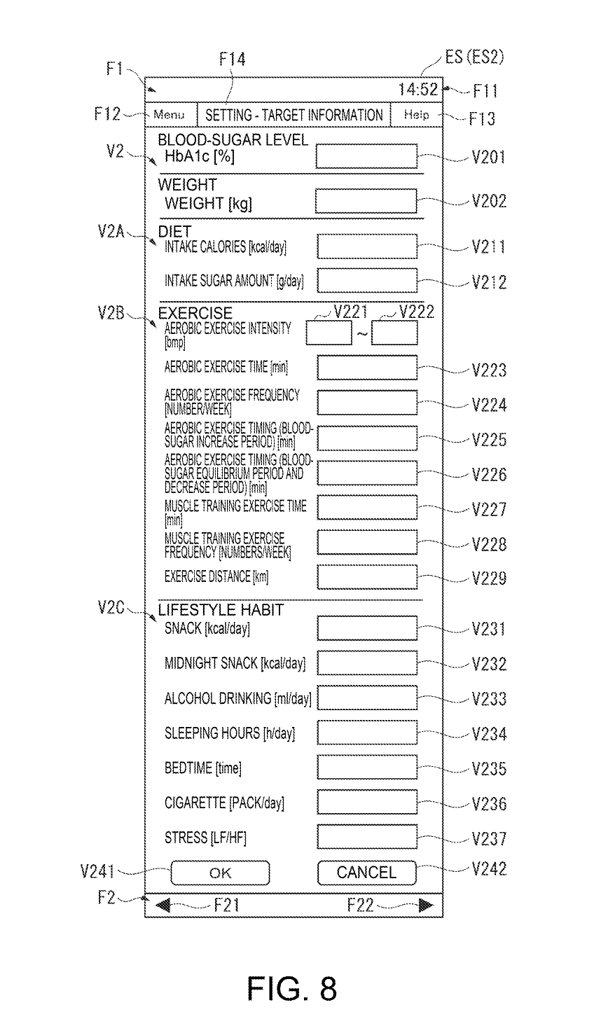

When the registration button V113 is pressed, input content of the entry fields V101 to V112 is acquired by the information acquisition unit 375 and are stored in the storage unit 35 by the information registration unit 376. Thereafter, in the embodiment, the screen transitions to a target information registration screen ES2 (see FIG. 8) to be described below.

The BMI can be calculated based on height and weight, as described above. Therefore, when height and weight are input to the height entry field V107 and the weight entry field V108, the BMI based on the input height and weight is set in the BMI entry field V109.

When the input content is registered, the physique included in the initial setting information is decided based on the set BMI and registered.

Further, the value of HOMA-β which is an index of the insulin secretory capacity and the value of HOMA-R which is an index of the insulin resistance can be calculated based on the CPR value and the IRI value input to the CPR value entry field V111 and the IRI value entry field V112. One of “insulin secretory capacity abortive type” and “insulin resistance type” is decided and registered as the type of diabetes included in the initial setting information based on the values of HOMA-β and HOMA-R.

On the other hand, when the cancel button V114 is pressed, the screen returns to the screen (for example, the menu screen) displayed immediately before the initial setting information registration screen ES1.

### Target Information Registration Screen

FIG. 8 is a diagram illustrating an example of the target information registration screen ES2.

The target information registration screen ES2 is a screen that is included in the execution screen ES and is a screen on which the target information is input and registered. As illustrated in FIG. 8, the fixed display regions F1 and F2 and a variable display region V2 are set in the target information registration screen ES2.

Entry fields V201 and V202 for inputting the target blood-sugar level and the target weight, entry fields V2A to V2C divided to the behaviors of the diet, the exercise, and the lifestyle habit, a registration button V241, and a cancel button V242 are provided in the variable display region V2.

Specifically, entry fields V211 and V212 for inputting the target intake calories and the target sugar amount are disposed in the entry region V2A for inputting the target information regarding the diet.

Entry fields V221 and V222 for inputting the target pulse rate lower limit and the target pulse rate upper limit are disposed in the entry region V2B for inputting the target information regarding the exercise. Entry fields V223 to V229 for inputting the target exercise time, the target number of exercises, the target exercise time in the increase period, the target exercise time in the decrease period, the target training time, the target number of trainings, and the target exercise distance, respectively, are disposed in the entry region V2B.

Entry fields V231 to V237 for inputting the target snack calories, the target midnight snack calories, the target alcohol amount, the target sleeping hours, the target bedtime, and the target number of packs, and the target stress value, respectively, are disposed in the entry region V2C for inputting the target information regarding the lifestyle habit. As described above, the target stress value can be set in advance, and thus the entry field V237 may not be provided.

When the registration button V241 is pressed, the input content of the entry fields V201, V202, V211, V212, V221 to V229, and V231 to V237 is acquired by the information acquisition unit 375 and is stored and registered in the storage unit 35 by the information registration unit 376.

On the other hand, when the cancel button V242 is pressed, the screen returns to the screen displayed immediately before the target information registration screen ES2.

### Exercise Result Presentation/Input Screen

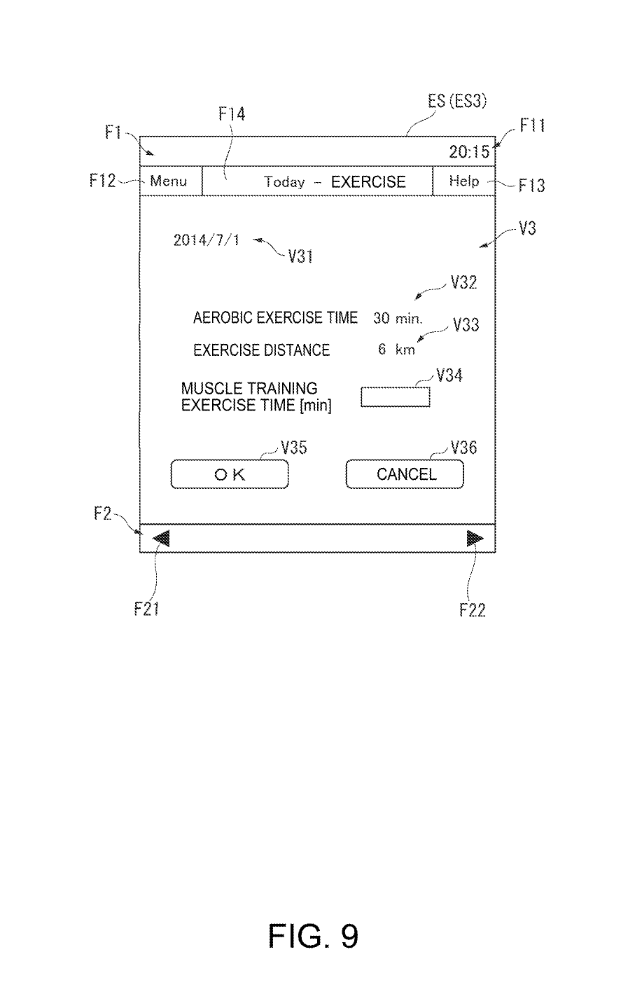

FIG. 9 is a diagram illustrating an example of an exercise result presentation/input screen ES3.

The exercise result presentation/input screen ES3 is a screen that is included in the execution screen ES and is a screen on which an analysis result of a behavior by the behavior analysis unit 377 based on the behavior information is presented and a time in which a muscle training is performed is input by the user. As illustrated in FIG. 9, the fixed display regions F1 and F2 and a variable display region V3 disposed between the fixed display regions F1 and F2 are set on the exercise result presentation/input screen ES3.

A date display field V31 for showing a date of that day, an exercise time display field V32 for displaying an aerobic exercise time (a sum time of an aerobic exercise per day) calculated by the behavior analysis unit 377, an exercise distance display field V33 for displaying an exercise distance at the time of the aerobic exercise calculated based on the positional information acquired at the time of the aerobic exercise by the behavior analysis unit 377, and an entry field V34 for inputting a time of a muscle training are set in the variable display region V3. The aerobic exercise time and the exercise distance set in the display field V33 are calculated by the behavior analysis unit 377. Further, a registration button V35 and a cancel button V36 are disposed in the variable display region V3.

When the registration button V35 is pressed, the input content to the entry field V34, that is, the time of the muscle training of that day, is acquired by the information acquisition unit 375 and is stored in the storage unit 35 by the information registration unit 376.

On the other hand, when the cancel button V36 is pressed, the screen returns to the screen displayed immediately before the exercise result presentation/input screen ES3.

### Physical Information Input Screen

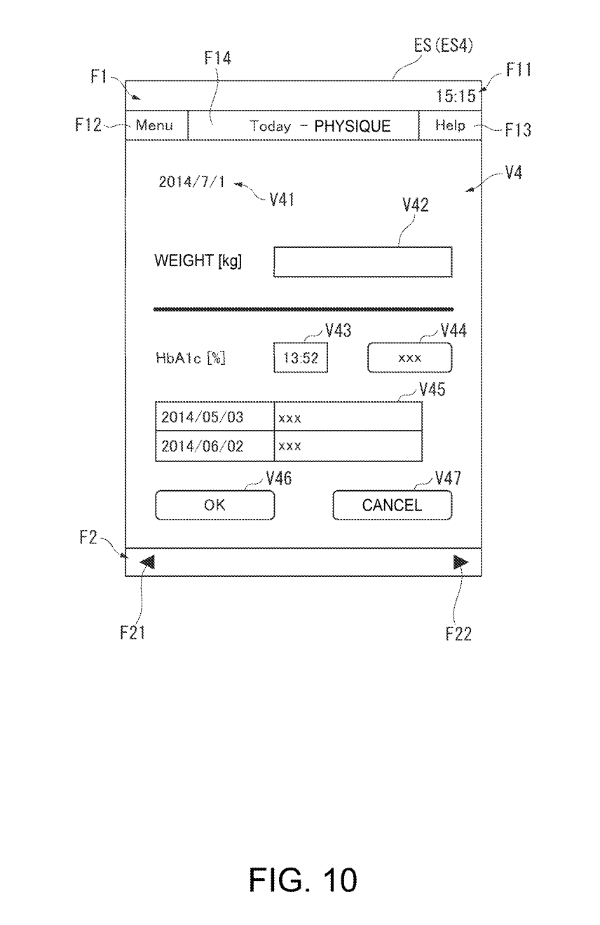

FIG. 10 is a diagram illustrating an example of the physical information input screen ES4.

The physical information input screen ES4 is a screen that is included in the execution screen ES and is a screen on which a weight and a blood-sugar level are input by the user. As illustrated in FIG. 10, the fixed display regions F1 and F2 and a variable display region V4 are set in the physical information input screen ES4.

A date display field V41 for showing a date of that day, an entry field V42 for inputting weight, entry fields V43 and V44 for inputting a measurement time of HbA1C and the value of HbA1C, a display field V45 for showing a previous measurement result of HbA1C, a registration button V46, and a cancel button V47 are provided in the variable display region V4.

When the registration button V46 is pressed, the input content to the entry fields V42 to V44 is stored in the storage unit 35 by the information registration unit 376, as described above.

On the other hand, when the cancel button V47 is pressed, the screen returns to the screen displayed immediately before the physical information input screen ES4.

### Diet Registration Screen

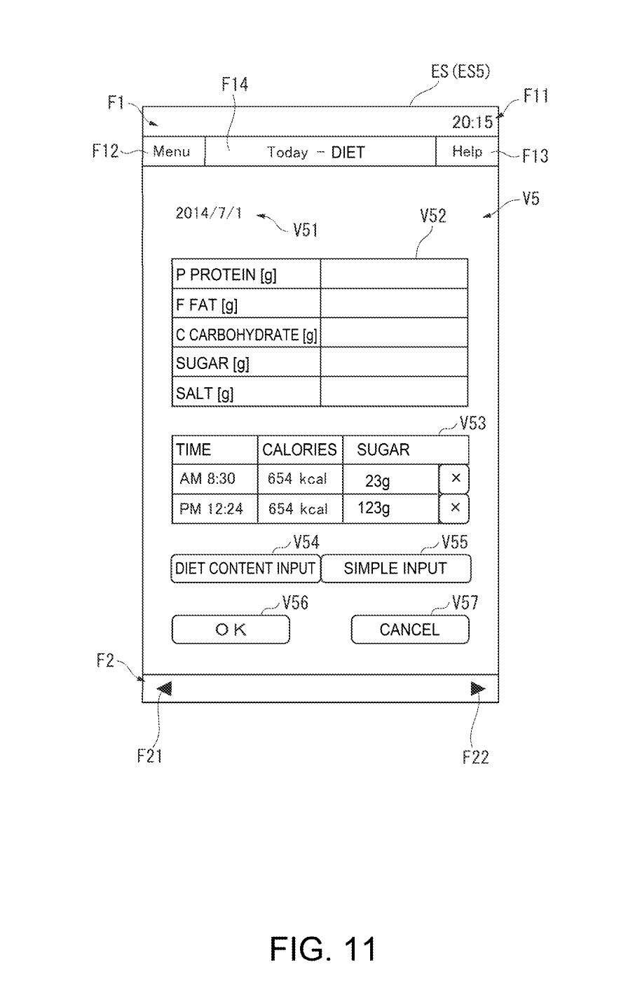

FIG. 11 is a diagram illustrating an example of a diet content registration screen ES5.

The diet content registration screen ES5 is a screen that is included in the execution screen ES and is a screen on which the content of diet eaten by the user is registered and displayed. The fixed display regions F1 and F2 and a variable display region V5 are set in the diet content registration screen ES5.

A date display field V51 for showing a date of that day, a display field V52 for displaying diet content of that day, a display field V53 for displaying an overview of the diet of that day, and a detail setting button V54, and a simple setting button V55, a registration button V56, and a cancel button V57 are disposed in the variable display region V5.

Of these fields, intake amounts of protein (P), fat (F), carbohydrate (C), sugar, and salt in that day are set in the display field V52.

The start time of the diet in that day and the intake calories and the sugar amount of the diet are set in the display field V53.

When the detail setting button V54 is pressed, although not illustrated, a screen on which a menu of the eaten diet, a diet amount, and a start time of the diet can be selected by the user is displayed. When the user selects and inputs the items according to the diet content on this screen, the diet content is analyzed, and the intake amounts of protein, fat, carbohydrate, sugar, and salt in the diet and a total of calories of the diet are calculated. Then, the calculation result and the start time of the diet are reflected to the display fields V52 and V53. The diet content may be analyzed by querying an external database (a database retained in an external apparatus) or may be analyzed via an external server (external apparatus).

### Simple Setting Screen

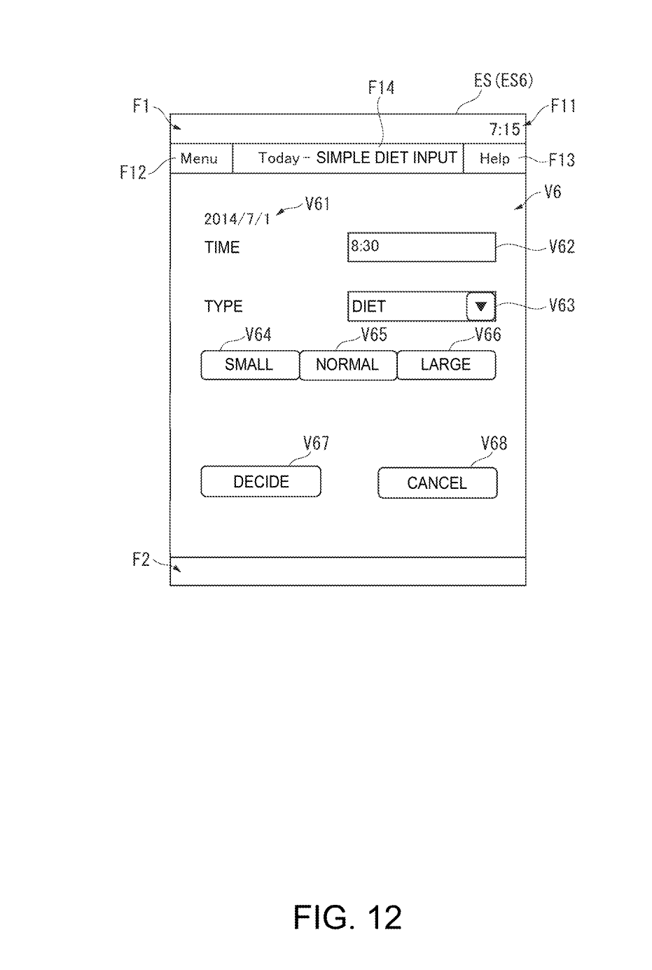

FIG. 12 is a diagram illustrating an example of the simple setting screen ES6.

On the other hand, when the simple setting button V55 is pressed, a display screen transitions to the simple setting screen ES6 illustrated in FIG. 12. The simple setting screen ES6 is a screen that is included in the execution screen ES and is a screen on which a start time, classification, and a diet amount (subjective information or relative information of the diet amount) of diet are registered. The fixed display regions F1 and F2 and a variable display region V6 are set in the simple setting screen ES6.

In the fixed display region F2 of the simple setting screen ES6, buttons F21 and F22 are not disposed, but the buttons F21 and F22 may be disposed as buttons for transitioning to another execution screen ES.

A date display field V61 for showing a date of that day, an entry field V62 for inputting a time of the diet, an entry field V63 for selecting the classification such as “diet,” “alcohol,” and “snack,” amount setting buttons V64 to V66 pressed according to the diet amount, a decision button V67, and a cancel button V68 are disposed in the variable display region V6.

Of these buttons, “small” is notated in the button V64, “normal” is notated in the button V65, and “large” is notated in the button V66. When any one of the buttons V64 to V66 is pressed, a diet amount, an alcohol amount, and a snack amount according to the pressed button are set.

Thereafter, when the decision button V67 is pressed, the screen transitions to the diet content registration screen ES5 and the diet content input and selected on the simple setting screen ES6 is reflected to the display fields V52 and V53.

When the cancel button V68 is pressed, the diet content registration screen ES5 in the state before the simple setting button V55 is pressed is displayed.

Referring back to FIG. 11, when the registration button V56 of the diet content registration screen ES5 is pressed, the content (the content set on the transitioned screen when the detail setting button V54 or the simple setting button V55 is pressed) set in the display field V52 is stored in the storage unit 35 by the information registration unit 376, as described above.

When the cancel button V57 is pressed, the screen returns to the screen displayed immediately before the diet content registration screen ES5.

### Sleeping Hours Registration Screen

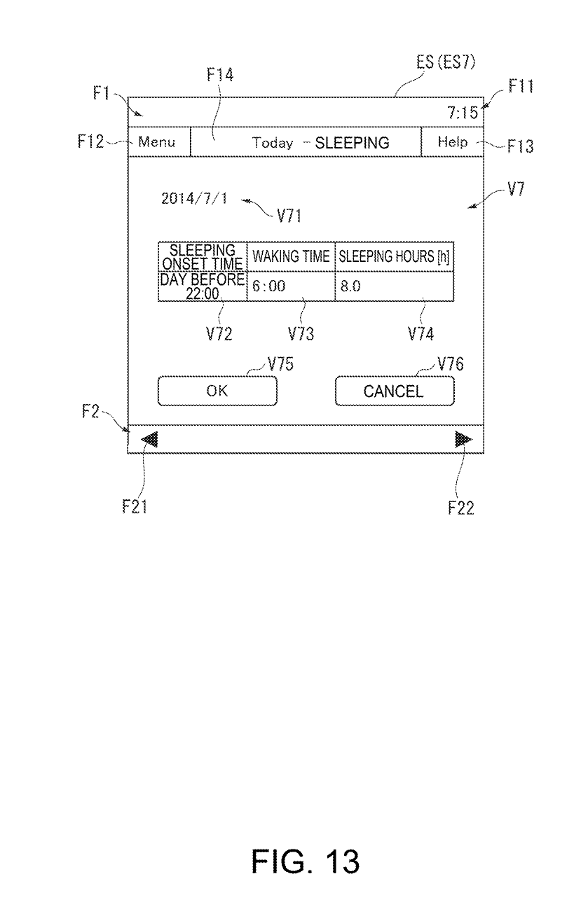

FIG. 13 is a diagram illustrating an example of the sleeping hours registration screen ES7.

The sleeping hours registration screen ES7 is a screen that is included in the execution screen ES and is a screen on which sleeping hours are input by the user. As illustrated in FIG. 13, the fixed display regions F1 and F2 and a variable display region V7 are set in the sleeping hours registration screen ES7.

A date display field V71 for showing a date of that day, a sleeping onset time setting field V72, a rising time setting field V73, a sleeping hours display field V74, a registration button V75, and a cancel button V76 are disposed in the variable display region V7.

Of these fields, a sleeping onset time and a rising time determined by the behavior analysis unit 377 are set in the sleeping onset time setting field V72 and a rising time setting field V73, respectively. The sleeping onset time setting field V72 and the rising time setting field V73 are also entry fields. For example, when the behavior information may not be acquired, the user can also correct each time.

In the sleeping hours display field V74, the sleeping hours of the user are set based on the times set and input in the setting fields V72 and V73.

When the registration button V75 is pressed, the content set in the setting fields V72 and V73 and the sleeping hours display field V74 is stored in the storage unit 35 by the information registration unit 376, as described above.

On the other hand, when the cancel button V76 is pressed, the screen returns to the screen displayed immediately before the sleeping hours registration screen ES7.

### Lifestyle Content Registration Screen

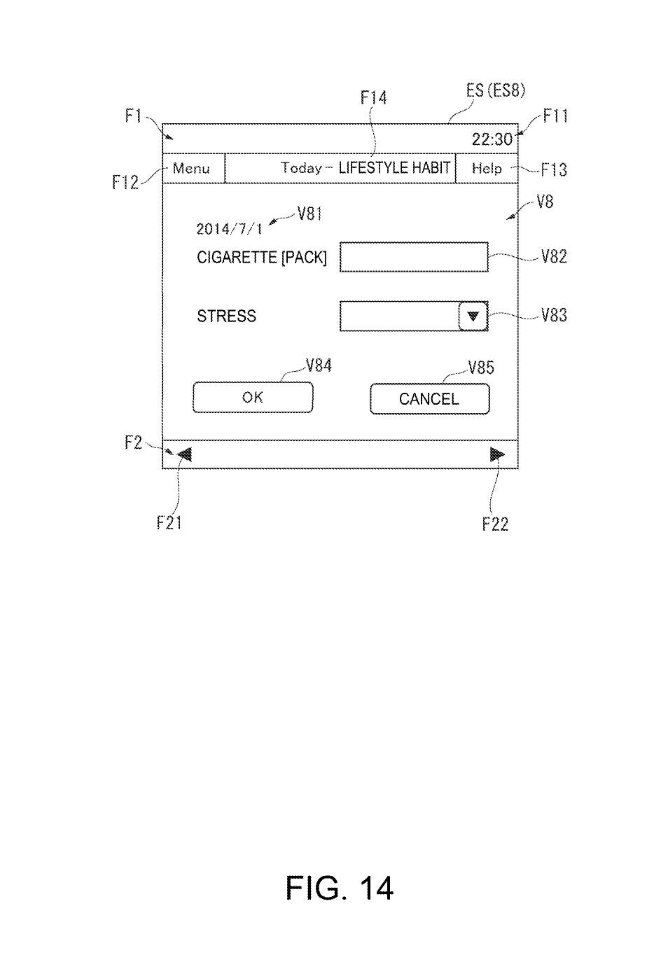

FIG. 14 is a diagram illustrating an example of the lifestyle content registration screen ES8.

The lifestyle content registration screen ES8 is a screen that is included in the execution screen ES and is a screen on which content regarding the lifestyle is input by the user. The “alcohol drinking” included in the lifestyle above is registered on the diet content registration screen ES5.

As illustrated in FIG. 14, the fixed display regions F1 and F2 and a variable display region V8 are set in the lifestyle content registration screen ES8.

A date display field V81 for showing a date of that day, an entry field V82 for inputting the number of cigarettes smoked in that day, an entry field V83 for selecting a stress state of the user, a registration button V84, and a cancel button V85 are disposed in the variable display region V8.

Of the fields, the entry field V83 is a selection field for selecting the stress state of the user from among “stress state,” “normal,” and “relaxed state.” The stress state of the user can be determined from the value of LF/HF based on biometric information (a detection result of a pulse wave) included in the behavior information. Therefore, when the behavior information is acquired, the entry field V83 is considered not to be displayed.

When the registration button V84 is pressed, the content set in the entry fields V82 and V83 is stored in the storage unit 35 by the information registration unit 376, as described above.

On the other hand, when the cancel button V85 is pressed, the screen returns to the screen displayed immediately before the lifestyle content registration screen ES8.

### Exercise Input Screen

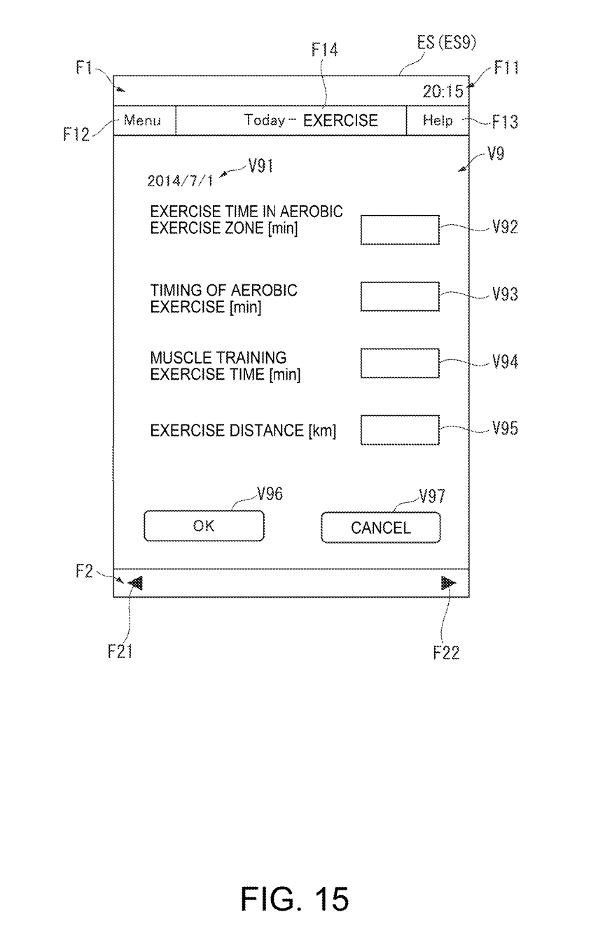

FIG. 15 is a diagram illustrating an example of the exercise input screen ES9.

Here, when the information processing apparatus 3 does not acquire the behavior information from the detection apparatus 2, not only an execution time of the muscle training but also an exercise time of the aerobic exercise and an execution time of the aerobic exercise after diet (start of the diet) may not be acquired. Therefore, when the behavior information is not acquired, the exercise input screen ES9 illustrated in FIG. 15 is displayed instead of the exercise result presentation/input screen ES3.

The exercise input screen ES9 is a screen that is included in the execution screen ES and is a screen on which an exercise state of that day is input by the user. As illustrated in FIG. 15, the fixed display regions F1 and F2 and a variable display region V9 are set in the exercise input screen ES9.

A date display field V91 for showing a date of that day, an entry field V92 for inputting an exercise time of an aerobic exercise performed on that day, an entry field V93 for inputting an elapsed time from the start of diet when the aerobic exercise starts, an entry field V94 for inputting a time of a muscle training of that day, and an entry field V95 for inputting an exercise distance of that day are set in the variable display region V9. Further, a registration button V96 and a cancel button V97 are disposed in the variable display region V9.

When the registration button V96 is pressed, the content set in the entry fields V92 to V95 is stored in the storage unit 35 by the information registration unit 376, as described above.

On the other hand, when the cancel button V97 is pressed, the screen returns to the screen displayed immediately before the exercise input screen ES9.

### Result Presentation Screen

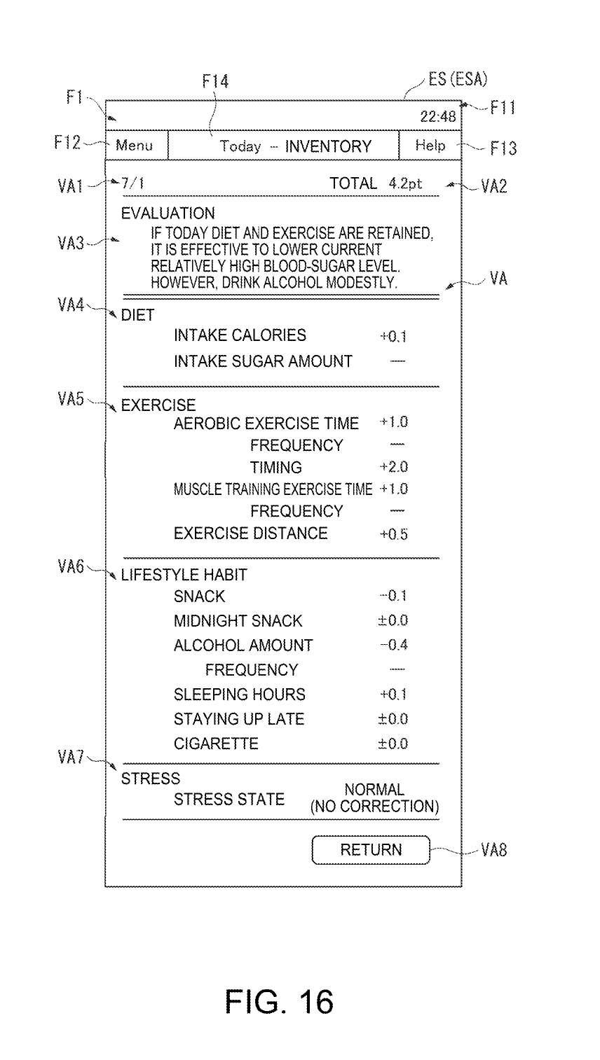

FIG. 16 is a diagram illustrating an example of the result presentation screen ESA.

When a manipulation signal is input in response to a predetermined input manipulation from the manipulation unit 31, the presented information generation unit 379 causes the display control unit 372 to draw the result presentation screen ESA illustrated in FIG. 16 and causes the display unit 33 to display the result presentation screen ESA.

The result presentation screen ESA is a screen that is included in the execution screen ES and is a screen on which the sum value of the points calculated by the calculation unit 378 on a date designated by the user, the evaluation of the sum value, and presented information including the details of the sum value. As illustrated in FIG. 16, the fixed display regions F1 and F2 and a variable display region VA are set in the result presentation screen ESA.

A date display field VA1 for showing a date designated by the user, a sum value display field VA2, an evaluation display field VA3, detail display fields VA4 to VA7, and a return button VA8 for returning the screen to the immediately previous screen are disposed in the variable display region VA.

A sum value of the date designated by the user is set in the sum value display field VA2. Specifically, in the sum value display field VA2, a sum value of the points calculated by the calculation unit 378 is set on that date based on the calculation rule according to each of the classified items of a behavior determined from the acquired behavior information and a behavior input by the user. A difference between the sum value of that day and the sum value of the previous day may be further set.

In the evaluation display field VA3, evaluation regarding a physical condition of the user according to the sum value is set. Specifically, a daily sum value, daily transition of each item, and evaluation of the physical condition of the user based on the transition or the like of the blood-sugar level input by the user are set in the evaluation display field VA3. The evaluation is generated by the presented information generation unit 379.

The detail display fields VA4 to VA7 are display fields for showing the point details of the behavior corresponding to the sum value. The points set in the display fields VA4 to VA7 are points that the calculation unit 378 calculates based on the behavior of the user on that day, the items of the behavior (the diet, the exercise, and the lifestyle habit), and the calculation rule stored in advance according to stress.

Specifically, the detail display field VA4 is a display field for showing the details of the points in regard to “diet.” In the detail display fields VA4, display fields of the points of the items of “intake calories” and “intake sugar amount” are set. The points which the calculation unit 378 calculates for each item using the calculation rules are set based on the diet content of the user on that day are set in these display fields. For example, the points calculated using the calculation rule for the intake calories based on the intake calories of that day are set in the field of “intake calories.” The same applies to “intake sugar amount.”

The detail display field VA5 is a display field for showing the details of the points in regard to “exercise.” The display fields of the points of the items, “aerobic exercise time,” “aerobic exercise frequency,” “aerobic exercise timing,” “muscle training exercise time,” “muscle training exercise frequency,” and “exercise distance,” are set in the detail display field VA5. Of the display fields, the points which the calculation unit 378 calculates for each item using the calculation rules based on the exercise content of the user on that day are set in the display fields of “aerobic exercise time,” “aerobic exercise timing,” “muscle training exercise time,” and “exercise distance.” The points which the calculation unit 378 calculates using the calculation rules based on a frequency at which the aerobic exercise and the muscle training are performed in a week including that day by the user are set in the display fields of “aerobic exercise frequency” and “muscle training exercise frequency.”

The detail display field VA6 is a display field for showing the details of the points in regard to “lifestyle habit.” In the detail display field VA6, display fields of the points of the items, “snack,” “midnight snack,” “alcohol amount,” “alcohol drinking frequency,” “sleeping hours,” “staying up late,” and “cigarette” are set. Of the fields, the points which the calculation unit 378 calculates for each item using the calculation rule based on the item in regard to the lifestyle habit of the user on that day are set in the other display fields except for the display field of “alcohol drinking frequency.” In the display field of “alcohol drinking frequency,” the points according to the alcohol drinking frequency of the user in a week including that day are calculated and set using the calculation rule by the calculation unit 378. As described above, the points for the item of the alcohol drinking frequency are calculated once per week.

The detail display field VA7 is a display field indicating a stress state of the user and indicating whether to correct the sum value by the calculation rule according to the stress state.

Specifically, as the stress state of the user, the stress state of the user based on the behavior information indicates one of “stress state,” “normal,” and “relaxed state” when the behavior information is acquired. Further, the stress state of the user input on the lifestyle content registration screen ES8 indicates one of the same “stress state,” “normal,” and “relaxed state” when the behavior information is not acquired.

In regard to whether to correct the sum value, “correction” is indicated when the correction is executed, and “no correction” is indicated when the correction is not executed. As described in the calculation rule for the stress, as the case in which the correction of the sum value is executed, there is a case in which the value of LF/HF of the user based on the behavior information exceeds the target stress value or a case in which “stress state” is input as a user state of that day.

When the user confirms the result presentation screen ESA and confirms the displayed sum value, the user can comprehend whether a behavior of the user is effective in an improvement (for example, improvement of diabetes) of the physical condition and how effective the behavior is. When the user confirms the evaluation indicated in the evaluation display field VA3, the user can understand a physical condition of the user associated with diabetes included in the lifestyle habit diseases or a way to improve the physical condition although it is difficult for the user to comprehend what the sum value means. Further, when the user comprehends the points of the items set in the detail display fields VA4 to VA6, the user can comprehend which behaviors lower the points of the items, in other words, which items do not contribute to the improvement in the physical condition. Thus, it is possible to provide an opportunity to reconsider daily behaviors.

### Integrated Value Presentation Screen

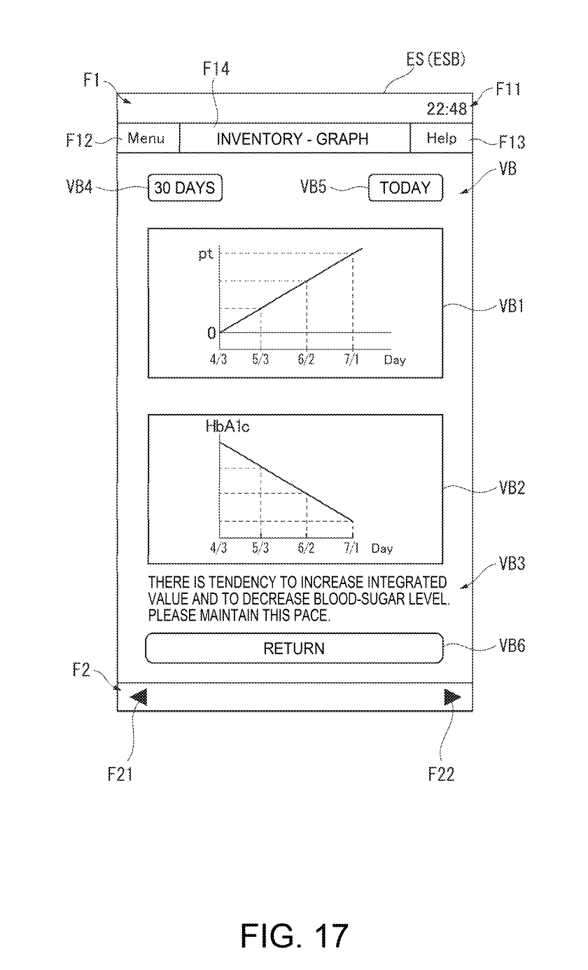

FIG. 17 is a diagram illustrating an example of an integrated value presentation screen ESB.

When a manipulation signal is input from the manipulation unit 31 in response to an input manipulation of displaying an integrated value within a predetermined period, the presented information generation unit 379 causes the display control unit 372 to draw the integrated value presentation screen ESB illustrated in FIG. 17 and causes the display unit 33 to display the integrated value presentation screen ESB.

The integrated value presentation screen ESB is a screen that is included in the execution screen ES and is a screen on which a change in the integrated value of the daily sum values within the predetermined period and a change in the blood-sugar level input on the physical information input screen ES4 within the predetermined period are shown by graphs and the presented information including the evaluation of the physical condition of the user according to the integrated value is also shown. Since the value of HbA1c (the concentration of HbA1c in blood) which is a blood-sugar control index indicates an average blood-sugar level before 30 days to 60 days from an inspection day, a duration of the previous 90 days from a current date is set as the predetermined period (retention period) in the embodiment so that a relation between the change in the value of HbA1c and the change in the integrated value can be comprehended.

As illustrated in FIG. 17, the fixed display regions F1 and F2 and a variable display region VB are set in the integrated value presentation screen ESB.

An integrated value graph display field VB1, a blood-sugar level graph display field VB2, an evaluation display field VB3, a display period change button VB4, a switch button VB5, and a return button VB6 for returning to an immediately previous screen are disposed in the variable display region VB.

In the integrated value graph display field VB1, a graph indicating the change in the integrated value of the sum values within the predetermined period is set.

In the blood-sugar level graph display field VB2, a graph indicating the change in the value of HbA1c based on the value of HbA1c (the value of HbA1c input on the physical information input screen ES4) measured within the predetermined period is set.

In the evaluation display field VB3, the evaluation of the physical condition of the user according to the integrated value is set. As the evaluation, evaluation of the change in the integrated value or evaluation of a current behavior of the user based on correlation between the change in the integrated value and the change in the blood-sugar level can be exemplified. As described above, the evaluation is generated by the presented information generation unit 379.

The display period change button VB4 is used to change a period of each graph set in each of the integrated value graph display field VB1 and the blood-sugar level graph display field VB2. For example, when the button VB4 is pressed, each graph for which the predetermined period is changed to the duration of the previous 30 days from the current date is set in each of the display fields VB1 and VB2. That is, when the display period change button VB4 is pressed, the predetermined period which is a period in which the sum values are integrated to obtain the integrated value is changed to the duration of the previous 30 days from the current date.

When the switch button VB5 is pressed, the result presentation screen ESA (see FIG. 16) of that day is displayed. Further, by changing the date, the result presentation screen ESA of the designated date can also be displayed.

### Classified Behavior Change Presentation Screen

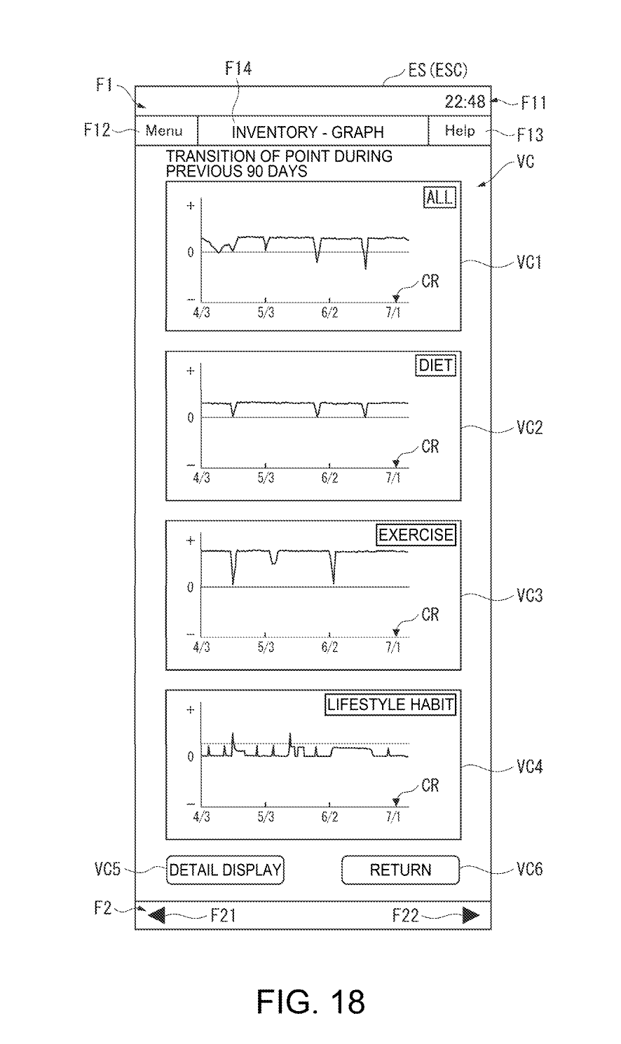

FIG. 18 is a diagram illustrating an example of the classified behavior change presentation screen ESC.

When a manipulation signal is input from the manipulation unit 31 in response to an input manipulation of displaying a graph indicating a change in a classified behavior sum value within the predetermined period (90 days), the presented information generation unit 379 causes the display control unit 372 to draw the classified behavior change presentation screen ESC illustrated in FIG. 18 and causes the display unit 33 to display the classified behavior change presentation screen ESC.

The classified behavior change presentation screen ESC is a screen that is included in the execution screen ES and is a screen on which not only a graph indicating transition of a sum value of a behavior of each day but also graphs indicating transition of sum values of “diet,” “exercise,” and “lifestyle habit” which are behaviors of each day are presented. As illustrated in FIG. 18, the fixed display regions F1 and F2 and a variable display region VC are set in the classified behavior change presentation screen ESC.

Four graph display fields VC1 to VC4, a detail display button VC5, and a return button VC6 for returning to the immediately previous screen are set in the variable display region VC.

In the graph display field VC1, a graph indicating daily transition of the sum value in “diet,” “exercise,” and “lifestyle habit” in the predetermined period is set.

In the graph display field VC2, a graph indicating daily transition of the sum value of each item of “diet” in the predetermined period is set.

In the graph display field VC3, a graph indicating daily transition of the sum value of each item of “exercise” in the predetermined period is set.

In the graph display field VC4, a graph indicating daily transition of the sum value of each item of “lifestyle habit” in the predetermined period is set.

By observing the graphs of the graph display fields VC1 to VC4, the user can comprehend which behavior of the item has a good influence or a bad influence on the physical condition. Thus, the user can consult with a medical worker about and how the user can behave to appropriately improve the physical condition.

In the classified behavior change presentation screen ESC, the detail display button VC5 is a button that is used to display, on the horizontal axis (the horizontal axis representing a date) of the graph set in each of the graph display fields VC1 to VC4, the result presentation screen ESA (see FIG. 16) of a date indicated by a cursor CR which can move along the horizontal axis. The cursor CR can be moved by the user in connection with the graph of each of the graph display fields VC1 to VC4.

Here, since the value of HbA1c input on the physical information input screen ES4 indicates the average blood-sugar value of the previous 30 to 60 days from an inspection date, the predetermined period is set to a duration of the previous 90 days from a current date and an integration period of the sum value at the time of the calculation of the integrated value is also set to a duration of the previous 90 days in conformity to the predetermined period. In the integrated value presentation screen ESB and the classified behavior change presentation screen ESC, the period on the horizontal axis of each graph also conforms to the predetermined period. However, the predetermined period is not limited thereto. For example, as described above, the value of glico albumin indicates an average blood-sugar value from the last month (particularly, most recently two weeks) of blood collection to blood collection. From this viewpoint, when a configuration in which the value of glico albumin is input instead of the value of HbA1c is adopted (when the value of glico albumin is adopted as an index indicating a diabetes state), the predetermined period can be set to a period equal to or greater than one previous week and equal to or less than two previous weeks from the current date, and thus the change in the integrated value can match the change in the blood-sugar value.

### Message Presentation Screen

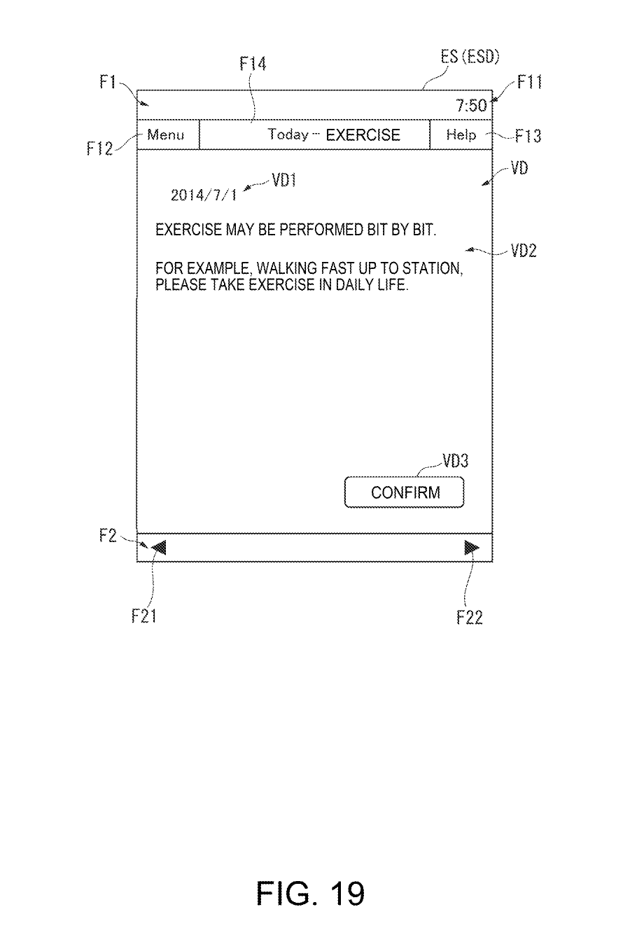

FIG. 19 is a diagram illustrating an example of the message presentation screen ESD.

The presented information generation unit 379 generates a message according to a generated event, causes the display control unit 372 to draw the message presentation screen ESD including the message, and causes the display unit 33 to display the message presentation screen ESD.

The message presentation screen ESD is a screen that is included in the execution screen ES and is a screen on which a predetermined message is presented to the user. As illustrated in FIG. 19, the fixed display regions F1 and F2 and a variable display region VD are set in the message presentation screen ESD.

A date display field VD1 indicating the date of that day, a message display field VD2, and a confirmation button VD3 are provided in the variable display region VD. When the confirmation button VD3 is pressed among the fields and the button, the message presentation screen ESD is closed.

In the message display VD2, a message (presented information) generated by the presented information generation unit 379 is displayed according to the generated event.

For example, when it is determined with reference to registered content of several previous days that an aerobic exercise is insufficient, the presented information generation unit 379 generates a message prompting exercise and displays the message presentation screen ESD including the message. Likewise, even when it is determined with reference to registered content of several previous days that exercise is not performed, the presented information generation unit 379 displays the message presentation screen ESD including another message prompting exercise.

Although not illustrated, when diet content of that day is registered on the diet content registration screen ES5 and the intake sugar amount per day at the time of registration of the diet content is determined to exceed 80% of the target sugar amount, the presented information generation unit 379 generates a message indicating that the intake sugar amount exceeds 80% of the target sugar amount and displays the message presentation screen ESD including this message. Even when the intake calories per day exceed 80% of the target intake calories, the presented information generation unit 379 displays the message presentation screen ESD including the same message. Further, as a ratio of the intake sugar amount to the target sugar amount and a ratio of the intake calories to the target intake calories increase, the presented information generation unit 379 displays the message presentation screen ESD including a message indicating warning implication.

Although not illustrated, when exercise is determined to be performed based on the behavior information received from the detection apparatus 2 or an exercise time of an aerobic exercise input on the exercise input screen ES9 is an exercise time in which the points occur based on the calculation rules, the presented information generation unit 379 displays the message presentation screen ESD including a message indicating that the points are added. The same applies to a case in which an execution time of a muscle training is an execution time in which the points occur.

When the aerobic exercise frequency per week is a frequency at which the points occur based on the calculation rule, the presented information generation unit 379 displays the message presentation screen ESD including a message indicating that the points are added.

Thus, the presented information generation unit 379 displays the message presentation screen ESD at a timing at which exercise by which the points are added is performed, so that the user can recognize that the points are added, that is, a behavior by which the physical condition is improved is performed, and thus the user can have a desire to perform the behavior.

On the other hand, the presented information generation unit 379 displays the message presentation screen ESD at a timing at which an attention is called, so that the user can recognize that a behavior by which the physical condition degrades is performed, and thus the user can abstain from the behavior.

### Advantages of First Embodiment

In the physical condition presentation system 1 according to the above-described embodiment, the following advantages can be obtained. The calculation unit 378 calculates the points based on the calculation rule in regard to the item classified to the behavior performed within the predetermined period by the user. Then, the result presentation screen ESA and the integrated value presentation screen ESB are displayed in which the presented information including the graphs indicating the sum value of the calculated points and the integrated value and the evaluation of the physical condition according to the sum value of the points and the integrated value is set.

Accordingly, the content of the behavior connected with the index indicating the physical condition of the user can be digitized. In other words, the change in the value of HbA1c which is the blood-sugar control index can be connected with the change in the points (the sum value and the integrated value) calculated according to the behavior related to the change in the blood-sugar level. Therefore, when the points (the sum value and the integrated value) are displayed and the user comprehend the points, the user can simply comprehend whether the behavior for improving the physical condition is performed. By presenting the evaluation of the physical condition according to the points, it is possible to comprehend the evaluation of the physical condition obtained by performing the behavior in more detail.

Since the calculation rule is a rule by which the points are added as the user performs a behavior in an improvement direction of the physical condition, the increase in the points can lead to increase a motivation of the behavior for improving the physical condition.

Since the calculation unit 378 calculates the sum value of the points per day and calculates the integrated value in the predetermined period, it is possible to suppress erroneous calculation more than when the integrated value of the points is calculated once according to the behavior performed in the predetermined period. Further, by comprehending the change in the sum value calculated per day, it is possible to look back on the daily behavior in consideration of improvement of the physical condition.

Since the physical condition is changed in an accumulation manner of the daily behavior, the integrated value can be calculated and evaluated according to the change in the actual physical condition of the user by presenting the integrated value of the points integrated in the predetermined period and the evaluation of the physical condition according to the integrated value.

Here, as described above, a ratio in which a health state associated with lifestyle habit diseases depends on the behavior content of the daily life is high.

Therefore, by adopting an index (specifically, an index indicating a diabetes state) indicating a health state related to lifestyle habit disease as the index indicating the physical condition and setting the calculation rule for each item included in the behaviors related to the lifestyle habit diseases in the daily life of the user, it is possible to digitize the behavior content of the user related to the lifestyle habit diseases. Thus, it is possible to calculate and evaluate the points according to the health state related to the lifestyle habit diseases of the user.

In the embodiment, the index indicating the physical condition of the user is an index indicating a diabetes state. The calculation rules set according to the items contributing to prevention, improvement, and remedy for diabetes among the items classified to the behaviors of the user are adopted. Accordingly, the calculation unit 378 can calculate integrated value of the points according to the diabetes state. Thus, even when the user merely confirms the presented integrated value or evaluation, the user can comprehend the diabetes state.

Here, the value of HbA1c is generally used as an index indicating a diabetes state. A measurement method and a determination method were also established. Hence, in the physical condition presentation system 1, HbA1c is adopted as an index indicating a diabetes state. Accordingly, the integrated value and the diabetes state can match each other relatively easily. The same applies even when glico albumin is adopted instead of HbA1c.

Based on the value of HbA1c, the average blood-sugar level up to blood collection from two months before of the blood collection can be determined. Therefore, in the physical condition presentation system 1 in which the value of HbA1c is adopted as the index indicating the diabetes state, the predetermined period is set to be a duration of the previous 90 days from the current date.

Accordingly, the period (the predetermined period) in which the integrated value is calculated can match a determination period of the average blood-sugar level. Furthermore, the change in the integrated value can match the change in the blood-sugar level. Thus, since the integrated value is presented and the user can confirm the daily change in the integrated value, the user can comprehend the condition of diabetes more easily.

Based on glico albumin, the average blood-sugar level from the last month (particularly, most recently two weeks) of blood collection to blood collection can be determined. Therefore, when glico albumin is adopted as the blood-sugar level control index, the predetermined period can be set to a period equal to or greater than one previous week and equal to or less than two previous weeks from the current date, and thus it is possible to obtain the same advantages as those when HbA1c is adopted.

Here, examples of prevention, improvement, and remedy for lifestyle habit diseases include dietary therapy and exercise therapy, but a disorder of lifestyle habit is related to progress of lifestyle habit diseases.

For this reason, behaviors included in the daily life, diet, exercise, and lifestyle habit are set as behaviors of the users for which the points are calculated by the calculation unit 378. Accordingly, the calculation unit 378 can appropriately calculate the integrated value including the points of the items classified to the behaviors related to the lifestyle habit diseases. Thus, when evaluation related to the transition of the integrated value and a state of a lifestyle habit disease (diabetes) is presented, comprehensive evaluation including the diet, the exercise, and the lifestyle habit can be presented to the user rather than separate evaluation of an execution state of the items included in the diet, the exercise, and the lifestyle habit.

It is troublesome for the user to determine whether exercise performed by the user is exercise of intensity and an exercise amount sufficient for improvement of diabetes.

On the other hand, the calculation unit 378 calculates the points in regard to exercise based on the behaviors of the user indicated by the behavior information acquired from the detection apparatus 2 by the information acquisition unit 375 and the calculation rules stored in the storage unit 35. Accordingly, it is not necessary to determine whether the content is exercise content (for example, intensity and an exercise amount of the exercise) performed by the user and such determination can be appropriately executed. Thus, it is possible to appropriately calculate the points of the behaviors classified to the exercise, and it is possible to more appropriately calculate the integrated value.

Since the detection apparatus 2 is configured to include a pulsimeter measuring a pulse rate, it is possible to more appropriately comprehend the exercise content (for example, an exercise amount and exercise intensity) of the user based on the pulse rate. Thus, the calculation unit 378 can more appropriately calculate the points of the items classified to the exercise and further more appropriately calculate the integrated value.

Here, the physical condition is changed according to a daily stress state of the user. For this reason, the calculation unit 378 can calculate the integrated value more suitable for the physical condition of the user by correcting the sum value of the points per day according to the stress state of the user on that day. Thus, the integrated value can be appropriately connected to the index, and thus the appropriate evaluation can be presented to the user.

The exercise has an effect on improvement in both of a physical state and a mental state. On the other hand, in regard to symptom of diabetes, although there is a limit, there is a tendency to increase the degree of improvement as an exercise distance or a load at the time of exercise is larger. Therefore, since the points are added based on the exercise distance of the user in the physical condition presentation system 1, not only is the user prompted to perform exercise but also the points can be appropriately calculated for the performed exercise.

Since the detection apparatus 2 is mounted on the user and detects the behavior information, the detection apparatus 2 is necessarily small-sized and lightweight so that a behavior of the user is interrupted.

On the other hand, the detection apparatus 2 which is the first apparatus includes the detection unit 21 and the information processing apparatus 3 which is the second apparatus configured as a smartphone or the like includes the display unit 33, the audio output unit 34, and the control unit 37 including the display control unit 372, the audio output control unit 373, the calculation unit 378, and the presented information generation unit 379. Accordingly, the detection apparatus 2 can be easily configured to be small-sized and lightweight. Thus, the detection apparatus 2 can be configured not to interrupt a behavior of the user.

Since the calculation unit 378 is included in the information processing apparatus 3 and the information processing apparatus 3 is not necessarily mounted on the user, a processing circuit such as a CPU with relatively large dimensions can be mounted. Therefore, the processing circuit can realize the functions of the calculation unit 378. Therefore, even when complicated calculation rules are adopted, the calculation unit 378 can reliably execute processes.

Likewise, since a display unit having a relatively large screen can be adopted as the display unit 33 displaying the presented information, it is possible to easily confirm the screens ESA to ESD including the presented information.

### Second Embodiment

Next, a second embodiment of the invention will be described.

A physical condition presentation system according to the embodiment has the same configuration as the physical condition presentation system 1 and is different from the physical condition presentation system 1 in that a detection apparatus presents the presented information to a user. In the following description, the same reference numerals are given to the same or substantially the same as the above-described portions and the description thereof will be omitted.

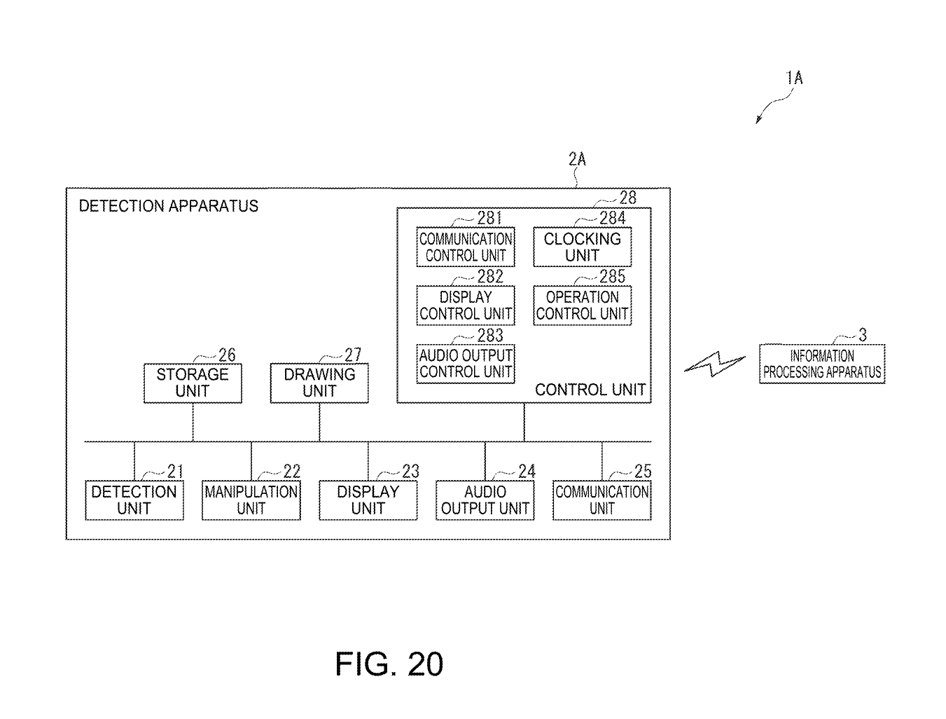

FIG. 20 is a block diagram illustrating a physical condition presentation system 1A according to the embodiment.

The physical condition presentation system 1A according to the embodiment includes a detection apparatus 2A, instead of the detection apparatus 2, and an information processing apparatus 3.

### Configuration of Information Processing Apparatus

In the embodiment, the information processing apparatus 3 corresponds to the second apparatus and the presented information output apparatus according to the invention. As in the information processing apparatus 3 of the physical condition presentation system 1, the information processing apparatus 3 calculates the points based on behavior information received from the detection apparatus 2A and information input by the user and evaluates a physical condition according to the points. Further, in the information processing apparatus 3, a communication unit 32 transmits presented information including the content of the evaluation and the points to the detection apparatus 2A. The information processing apparatus 3 includes a display unit 33 and an audio output unit 34. Thus, the display unit 33 and the audio output unit 34 present the presented information to the user on the screens ESA to ESD respectively illustrated in FIGS. 16 to 19. In the embodiment, however, the display unit 33 and the audio output unit 34 may not be included.

### Configuration of Detection Apparatus

As in the detection apparatus 2, the detection apparatus 2A detects and acquires the behavior information of the user on which the detection apparatus 2A is mounted, and then transmits the behavior information to the information processing apparatus 3. The detection apparatus 2A presents the presented information received from the information processing apparatus 3. In the embodiment, the detection apparatus 2A corresponds to the first apparatus according to the invention and can also be referred to as an information presentation apparatus.

As illustrated in FIG. 20, the detection apparatus 2A includes a detection unit 21, a manipulation unit 22, a display unit 23, an audio output unit 24, a communication unit 25, a storage unit 26, a drawing unit 27, and a control unit 28 controlling operations of these units.

The detection unit 21 detects and acquires the behavior information of the user, as described above. Specifically, the detection unit 21 includes a biometric information detection unit, an operation information detection unit, and a positional information acquisition unit and outputs detection results and acquisition results obtained by these units to the control unit 28.

The biometric information detection unit includes a pulse wave sensor that detects a pulse wave of the user and measures a pulse rate (a pulse rate per unit time) as biometric information based on the pulse wave detected by the pulse wave sensor.

The operation information detection unit includes an acceleration sensor and detects an acceleration value changing with an operation of the user.

The positional information acquisition unit includes a reception module that can receive a satellite signal transmitted from a positional information satellite such as a GPS satellite and acquires positional information indicating a current position based on the received satellite signal.

The detection apparatus 2A according to the embodiment includes the biometric information detection unit since that the detection apparatus 2A is a pulsimeter that detects a pulse rate of the user as the biometric information. However, when the detection apparatus 2A is an active amount meter, the control unit 28 to be described below calculates the number of steps and consumed calories of the user based on a change in the acceleration value detected by the operation information detection unit and user information such as height, weight, or the like input by the user. Therefore, the biometric information detection unit may not be included.

The manipulation unit 22 receives an input manipulation by the user and outputs manipulation information according to the input manipulation to the control unit 28. Examples of the manipulation information include manipulation information causing the behavior information to be transmitted to the information processing apparatus 3 and manipulation information causing the presented information generated by the information processing apparatus 3 to be displayed.

The display unit 23 displays an operation screen of the detection apparatus 2A and also displays a screen including the received presented information. As the display unit 23, various display panels such as the foregoing display unit 33 can be exemplified.

The audio output unit 24 is configured to include a speaker and outputs audio at the time of an operation of the detection apparatus 2A or audio related to the presented information. That is, in the embodiment, the audio output unit 24 forms the presentation unit according to the embodiment along with the display unit 23.

The communication unit 25 includes a communication module that can communicate with the information processing apparatus 3. The communication unit 25 transmits the behavior information generated by the control unit 28 to the information processing apparatus 3 and also outputs received information (for example, the presented information) to the control unit 28.

The storage unit 26 is configured by a storage device such as a flash memory and stores a program and data necessary for an operation of the detection apparatus 2A. For example, the storage unit 26 stores various kinds of information detected and acquired by the detection unit 21 and various kinds of information received from the information processing apparatus 3.

The drawing unit 27 includes a drawing circuit and draws an image (for example, an image of a screen including the operation screen or the presented information) to be displayed by the display unit 23.

The control unit 28 includes a processing circuit such as a CPU and controls an operation of the detection apparatus 2A. The control unit 28 includes a communication control unit 281, a display control unit 282, an audio output control unit 283, a clocking unit 284, and an operation control unit 285.

The communication control unit 281 controls the communication unit 25 to communicate with the information processing apparatus 3.

The display control unit 282 causes the drawing unit 27 to draw the image.

The audio output control unit 283 causes the audio output unit 24 to output audio information output by the audio output unit 24.

The clocking unit 284 clocks a current time.

The operation control unit 285 controls an operation of the detection apparatus 2A based on manipulation information input from the manipulation unit 22 or autonomously.

For example, as in the information registration unit 376, the operation control unit 285 stores the operation information (acceleration value) and the biometric information (pulse rate) detected by the detection unit 21 in the storage unit 26 in association with times at which the information is detected. When the user is determined to perform an aerobic exercise based on the biometric information and the operation information, the operation control unit 285 causes the detection unit 21 to acquire the positional information periodically and stores the acquired positional information in the storage unit 26 in association with a time at which the positional information is acquired. When the power consumption of the detection apparatus 2A is relatively small or a built-in battery capacity is relatively large, the operation control unit 285 may cause the detection unit 21 to acquire the positional information not only in a case in which the user performs the aerobic exercise but also in a case in which the user does not perform the aerobic exercise.

When operation information indicating that the behavior information is transmitted to the information processing apparatus 3 is input, the operation control unit 285 causes the communication control unit 281 and the communication unit 25 to transmit the behavior information including the biometric information, the operation information, and the positional information stored in the storage unit 26.

When presented information (for example, the image information of the screens ESA, ESB, ESC, and ESD respectively illustrated in FIGS. 16 to 19) to be presented to the user is received from the information processing apparatus 3 via the communication unit 25, the operation control unit 285 causes the drawing unit 27 to draw an image including the presented information and causes the display control unit 282 to display a corresponding screen on the display unit 23. At this time, when the received presented information includes audio information, the operation control unit 285 causes the audio output control unit 283 and the audio output unit 24 to output audio according to the audio information.

When the size of an image which can be displayed by the display unit 23 is small and the display unit 23 may not display the screens ESA to ESD at a time, a part of each of the screens ESA to ESD may be divided to be displayed or only a part of each of the screens ESA to ESD may be displayed.

In the former case, for example, the divided screen may be switched and displayed according to an input manipulation of the user on the manipulation unit 22 or all of the divided screens may be displayed through scrolling.

In the latter case, for example, only both or only one of the sum value of the points and the evaluation may be displayed on the result presentation screen ESA illustrated in FIG. 16. Alternatively, only a graph set in the integrated value graph display field VB1 may be displayed on the integrated value presentation screen ESB illustrated in FIG. 17.

Only a graph set in the graph display field VC1 may be displayed on the classified behavior change presentation screen ESC illustrated in FIG. 18. Alternatively, only a message set in the message display field VD2 may be displayed on the message presentation screen ESD illustrated in FIG. 19.

Alternatively, the information processing apparatus 3 may be configured to transmit only some of the screens ESA to ESD (for example, at least one of the sum value of the points and the evaluation on the result presentation screen ESA) as the presented information rather than transmitting all of the screens ESA to ESD as the presented information.

### Advantages of Second Embodiment

In the physical condition presentation system 1A according to the above-described embodiment, it is possible to obtain not only the same advantages as those of the physical condition presentation system 1 but also the following advantages.

The detection apparatus 2A mounted on the user includes the display unit 23 and the audio output unit 24 presenting the presented information received from the information processing apparatus 3 in addition to the detection unit 21. Accordingly, even when the information processing apparatus 3 is not carried with a hand, the user can confirm the presented information at a desired timing.

The information processing apparatus 3 communicating with the detection apparatus 2A includes the calculation unit 378 calculating the points and the presented information generation unit 379 generating each of the presented evaluation. Accordingly, such functions can be realized in a relatively large processing circuit which can be embedded in the information processing apparatus 3. Thus, even when a complicated calculation rule is adopted, it is possible to reliably calculate the points and evaluate the physical condition according to the points.

In the physical condition presentation system 1A, the information processing apparatus 3 transmits the presented information including the points (the sum value and the integrated value) and the evaluation of the physical condition according to the points to the detection apparatus 2A, and the detection apparatus 2A causes the display unit 23 and the audio output unit 24 to present the received presented information to the user. On the other hand, the information processing apparatus 3 may be configured to transmit the presented information including the points (the sum value and the integrated value) to the detection apparatus 2A, the detection apparatus 2A may be configured to evaluate the physical condition according to the received points and present at least one of the points and the evaluation.

### Third Embodiment

Next, a third embodiment of the invention will be described.

A physical condition presentation system according to the embodiment has the same configuration and functions as the physical condition presentation system 1 and is different from the physical condition presentation system 1 in that a detection apparatus generates the presented information and the information processing apparatus presents the presented information to a user. In the following description, the same reference numerals are given to the same or substantially the same as the above-described portions and the description thereof will be omitted.

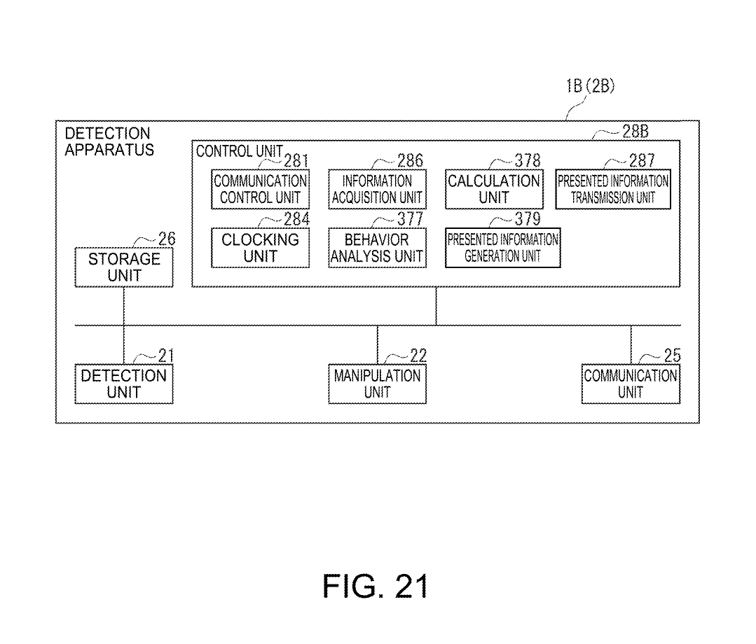

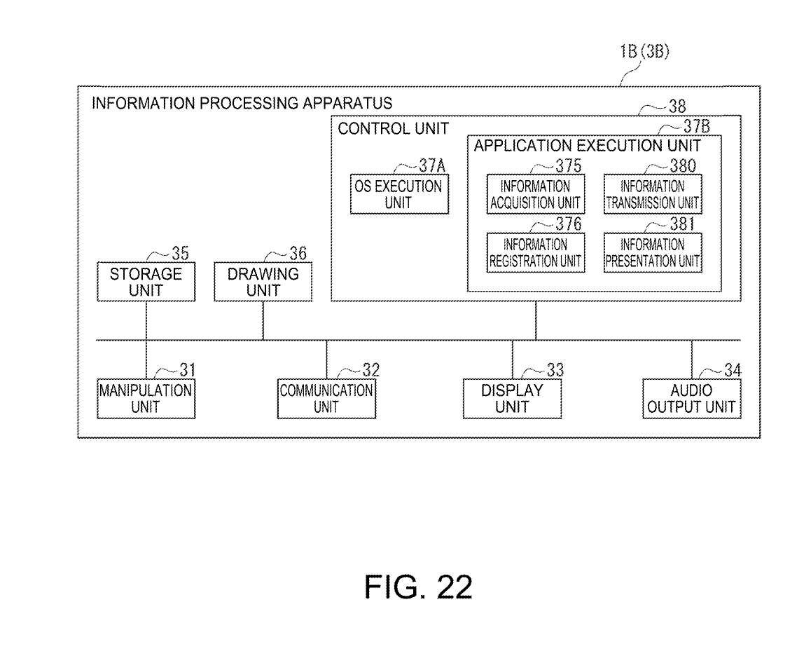

FIG. 21 is a block diagram illustrating the configuration of a detection apparatus 2B included in a physical condition presentation system 1B according to the embodiment. FIG. 22 is a block diagram illustrating the configuration of an information processing apparatus 3B included in the physical condition presentation system 1B.

The physical condition presentation system 1B according to the embodiment includes the detection apparatus 2B (see FIG. 21) and the information processing apparatus 3B (see FIG. 22), as illustrated in FIGS. 21 and 22.

### Configuration of Detection Apparatus

In the embodiment, the detection apparatus 2B corresponds to the first apparatus and the presentation information output apparatus according to the invention. As in the detection apparatuses 2 and 2A, the detection apparatus 2B detects and acquires the behavior information. Further, the detection apparatus 2B generates the presented information based on the behavior information, input information input by the user and received from the information processing apparatus 3B, and the calculation information, and then transmits (outputs) the presented information to the information processing apparatus 3B. As illustrated in FIG. 21, the detection apparatus 2B includes the detection unit 21, the manipulation unit 22, the communication unit 25, and the storage unit 26 described above and a control unit 28B controlling these units.

Of these units, the storage unit 26 stores the calculation information in advance in the embodiment, but the calculation information may be acquired from the information processing apparatus 3B.

As in the control unit 28, the control unit 28B controls an operation of the detection apparatus 2B. The control unit 28B includes the communication control unit 281 and the clocking unit 284 and includes an information acquisition unit 286, the behavior analysis unit 377, the calculation unit 378, the presented information generation unit 379, and a presented information transmission unit 287 functioning when the control unit 28B executes a presented information output program.

As in the operation control unit 285, the information acquisition unit 286 stores the biometric information (pulse rate) and the operation information (acceleration value) detected by the detection unit 21 in the storage unit 26 in association with times at which the information is detected. When the behavior analysis unit 377 determines that the user performs an aerobic exercise, the information acquisition unit 286 causes the detection unit 21 to acquire the positional information periodically and stores the acquired positional information in the storage unit 26 in association a time at which the positional information is acquired. The information acquisition unit 286 acquires input content to the manipulation unit 22. Further, the information acquisition unit 286 acquires information (for example, input information including the initial setting information input on the screens ES1 to ES9, target information, and information regarding a behavior or calculation information) received from the information processing apparatus 3B via the communication unit 25.

As described above, the behavior analysis unit 377 analyzes classification of a behavior performed by the user.

As described above, the calculation unit 378 calculates the sum value of the points according to the behavior of the user and the integrated value based on the acquired behavior information, the analysis result by the behavior analysis unit 377, and the calculation rule for each item.

As described above, the presented information generation unit 379 generates presented information including the calculated points (the sum value and the integrated value) and evaluation of the physical condition of the user according to the sum value of the points and the integrated value. In the embodiment, the presented information generation unit 379 executes evaluation and also generates image information of the screens ESA to ESD.

The presented information generated by the presented information generation unit 379 may include at least one of the points and the evaluation. Therefore, the presented information may not include the image information of the screens ESA to ESD.

The presented information transmission unit 287 causes the communication control unit 281 and the communication unit 25 to transmit the generated presented information to the information processing apparatus 3B.

### Configuration of Information Processing Apparatus

In the embodiment, the information processing apparatus 3B corresponds to the second apparatus according to the invention and is configured by a smartphone or the like, as in the information processing apparatus 3. As illustrated in FIG. 22, the information processing apparatus 3B has the same configuration and functions as those of the information processing apparatus 3 except that a control unit 38 is included instead of the control unit 37.

As in the control unit 37, the control unit 38 includes an OS execution unit 37A and an application execution unit 37B.

Of these units, the application execution unit 37B executes a physical condition presentation application different from the physical condition presentation application executed by the application execution unit 37B included in the control unit 37. That is, the application execution unit 37B displays the screens ES1 to ES9 and acquires input content on the screens ES1 to ES9, but does not execute a process such as calculation of the points and executes the physical condition presentation application presenting the presented information received from the detection apparatus 2B. The physical condition presentation application is stored in the storage unit 35.

The application execution unit 37B includes the information acquisition unit 375, the information registration unit 376, an information transmission unit 380, and an information presentation unit 381 functioning by executing the physical condition presentation application.

As described above, the information acquisition unit 375 acquires input information input by the user on the execution screens ES1 to ES9 displayed on the display unit 33 by the display control unit 372. The information acquisition unit 375 acquires presented information received from the detection apparatus 2B via the communication unit 32.

The information registration unit 376 stores the acquired input information and presented information in the storage unit 35.

The information transmission unit 380 causes the communication control unit 371 and the communication unit 32 to transmit the input information acquired and stored by the information acquisition unit 375 to the detection apparatus 2B in response to an input manipulation of the user on the manipulation unit 31 or a request from the detection apparatus 2B. As described above, the information transmission unit 380 may transmit the calculation information to the detection apparatus 2B in response to a request from the detection apparatus 2B.

The information presentation unit 381 presents the presented information received from the detection apparatus 2B and acquired by the information acquisition unit 375 to the user. Specifically, the information presentation unit 381 causes the display unit 33 to display the presented information and also causes the audio output unit 34 to output the present information, as necessary.

At this time, in the embodiment, the acquired present information is image information of the screens ESA to ESD including the points (the sum value and the integrated value) and the evaluation of the physical condition according to the points. Therefore, the information presentation unit 381 causes the drawing unit 36 to draw the images according to the image information, that is, the images of the screens ESA to ESD and displays the screens ESA to ESD. When only at least one of the points (the sum value and the integrated value) and the evaluation of the physical condition according to the points is included in the acquired presented information, the information presentation unit 381 causes the drawing unit 36 to draw an image including the presented information and causes the display unit 33 to display the images. Further, the information presentation unit 381 may cause the drawing unit 36 to draw (generate) the screens ESA to ESD including the presented information and display the screens ESA to ESD.

### Advantages of Third Embodiment

In the physical condition presentation system 1B according to the above-described embodiment, it is possible to obtain not only the same advantages as those of the physical condition presentation system 1 but also the following advantages.

The detection apparatus 2B includes the calculation unit 378 and the presented information generation unit 379, and the information processing apparatus 3B includes the information presentation unit 381 that presents the presented information generated and received by the presented information generation unit 379 to the user. Accordingly, compared to the information processing apparatus 3, the information processing apparatus 3B can be configured simply and the processes can be allotted to the detection apparatus 2B and the information processing apparatus 3B. Thus, it is possible to prevent a processing load of one of the detection apparatus 2B and the information processing apparatus 3B from being considerably increased more than that of the other.

The detection apparatus 2B does not include the display unit 23, the audio output unit 24, and the drawing unit 27, but may not include these units. In this case, the control unit 28B may have a configuration in which the display control unit 282 and the audio output control unit 283 are included, in addition to the foregoing configuration.

In the detection apparatus 2B, the calculation unit 378 calculates the points (the sum value and the integrated value), the presented information generation unit 379 evaluates the physical condition according to the points, and the presented information including the points and the evaluation is transmitted to the information processing apparatus 3B. However, the invention is not limited thereto, but the information processing apparatus 3B may be configured such that the physical condition is evaluated and presented according to the points included in the received presented information. That is, the information processing apparatus 3B may be configured to include the presented information generation unit 379.

The detection apparatus 2B receives the input information input in the information processing apparatus 3B and calculates the points according to the input information, the behavior information, and the calculation information. However, the invention is not limited thereto, but the input information and the calculation information may be input using the manipulation unit 22. In this case, when the detection apparatus 2B includes the display unit 23 and the display control unit 282 and the same screens as the screens ES1 to ES9 are displayed, an input manipulation can be easily executed.

### Fourth Embodiment

Next, a fourth embodiment of the invention will be described.

A physical condition presentation system according to the embodiment is different from the physical condition presentation system 1 in that one apparatus has the functions of the detection apparatus 2 and the information processing apparatus 3 and this apparatus is configured to communicate with a sever on a network via an external apparatus. In the following description, the same reference numerals are given to the same or substantially the same as the above-described portions and the description thereof will be omitted.

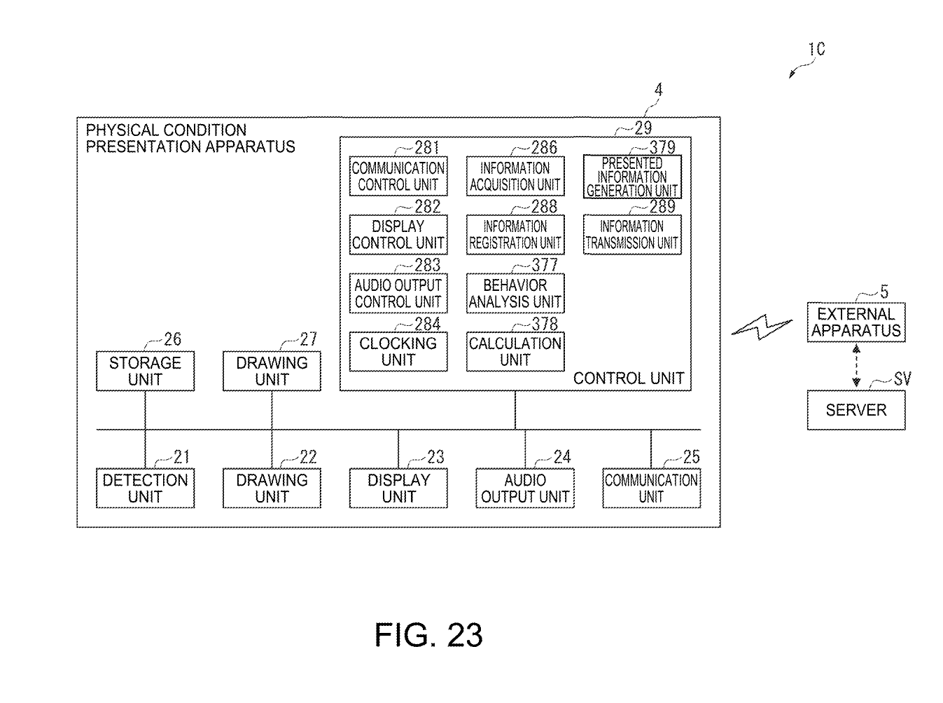

FIG. 23 is a block diagram illustrating the configuration of a physical condition presentation system 1C according to the embodiment.

The physical condition presentation system 1C according to the embodiment includes a physical condition presentation apparatus 4 and an external apparatus 5 and realizes the same functions as the physical condition presentation system 1, as illustrated in FIG. 23.

### Configuration of Physical Condition Presentation Apparatus

In the embodiment, the physical condition presentation apparatus 4 corresponds to the first apparatus and the presented information output apparatus according to the invention. The physical condition presentation apparatus 4 have the functions of the detection apparatus 2 and the information processing apparatus 3 and is mounted on a user to function. The physical condition presentation apparatus 4 includes the detection unit 21, the manipulation unit 22, the display unit 23, the audio output unit 24, the communication unit 25, the storage unit 26, the drawing unit 27, and a control unit 29.

Of these units, the storage unit 26 stores a physical condition presentation application and also includes the calculation information. The calculation information may be acquired from the external apparatus 5.

The control unit 29 includes a processing circuit such as a CPU and controls an operation of the physical condition presentation apparatus 4. The control unit 29 includes the communication control unit 281, the display control unit 282, the audio output control unit 283, and the clocking unit 284 and includes the information acquisition unit 286, an information registration unit 288, the behavior analysis unit 377, the calculation unit 378, the presented information generation unit 379, and an information transmission unit 289 functioning when the control unit 29 executes the physical condition presentation application (includes a presented information output program according to the invention) stored in the storage unit 26.

Of these units, the display control unit 282 causes the display unit 23 to display an operation screen of the physical condition presentation apparatus 4. For example, the display control unit 282 causes the display unit 23 to display the same screens as the screens ES1 to ES9.

The information acquisition unit 286 acquires input information (including the initial setting information, the target information, and the information regarding behaviors of the user) input by the user using the manipulation unit 22 on the same screens ES1 to ES9 displayed on the display unit 23. The information acquisition unit 286 acquires the behavior information (the biometric information, the operation information, the positional information, and the times at which the biometric information, the operation information, and the positional information are acquired) detected and acquired by the detection unit 21. Further, the information acquisition unit 286 acquires various kinds of information received from the external apparatus 5 via the communication unit 25.

The information registration unit 288 stores the various kinds of information acquired by the information acquisition unit 286 in the storage unit 26.

As described above, the behavior analysis unit 377 analyzes the acquired behavior information and analyzes the classification of the behavior performed by the user.

As described above, the calculation unit 378 calculates the points (the sum value and the integrated value) according to the behavior of the user based on the calculation information including the calculation rule for each item. At this time, when the calculation information is not stored in the storage unit 26, the calculation information may be acquired from the external apparatus 5 and the points may be calculated based on the acquired calculation information.

As described above, the presented information generation unit 379 generates the presented information including the calculated points (the sum value and the integrated value) and the evaluation of the physical condition of the user according to the points. The presented information generation unit 379 may acquire information (algorithm) necessary at the time of the generation of the presented information from the external apparatus 5. Then, the generated presented information is presented to the user by the display unit 23 and the audio output unit 24 under the control of the display control unit 282 and the audio output control unit 283, as described above.

The information transmission unit 289 transmits predetermined information to the external apparatus 5 via the communication control unit 281 and the communication unit 25. For example, when the information (for example, the calculation information) necessary to calculate the points or the information necessary to generate the presented information is acquired from the external apparatus 5, the information transmission unit 289 transmits request information to the external apparatus 5. When the behavior information or the presented information (the points and the evaluation) is transmitted to the server SV to be registered, the information transmission unit 289 transmits the behavior information or the presented information to the server SV via the external apparatus 5.

### Configuration of External Apparatus

In the embodiment, the external apparatus 5 corresponds to the second apparatus according to the invention and communicates with the physical condition presentation apparatus 4 to assist the physical condition presentation apparatus 4. The external apparatus 5 executes a process according to the request information received from the physical condition presentation apparatus 4. For example, the external apparatus 5 transmits the calculation information or the like to the physical condition presentation apparatus 4 according to the received request information. When the behavior information, the input information, or the presented information is received along with the request information indicating transmission to the server SV, the external apparatus 5 transmits the information to the server SV to register the information. That is, the external apparatus 5 relays the communication between the physical condition presentation apparatus 4 and the server SV.

In the embodiment, the external apparatus 5 can be configured by a smartphone or the like. The external apparatus 5 and the physical condition presentation apparatus 4 perform communication in conformity to a short-range wireless communication standard (for example, Bluetooth (registered trademark) or Zigbee (registered trademark)). The external apparatus 5 and the server SV perform communication in conformity to a wired communication standard or another wireless communication standard. However, the invention is not limited thereto, but the apparatuses and the server may perform communication in conformity to the same communication standard.

### Advantages of Fourth Embodiment

In the physical condition presentation system 1C according to the above-described embodiment, it is possible to obtain not only the same advantages as those of the physical condition presentation system 1 but also the following advantages.

The physical condition presentation apparatus 4 has the functions of the detection apparatus 2 and the information processing apparatus 3. Therefore, one physical condition presentation apparatus 4 can obtain the same advantages as the advantages obtained in the physical condition presentation system 1.

In the physical condition presentation system 1C, the function of the physical condition presentation apparatus 4 can be expanded by the external apparatus 5 assisting the physical condition presentation apparatus 4.

### Modifications of Embodiments

The invention is not limited to the foregoing embodiments, but modifications, improvements, and the like are included in the scope in which the objectives of the invention can be achieved.

In each of the foregoing embodiments, HbA1c and glico albumin which are the index indicating the diabetes state have been adopted as the index indicating the physical condition of the user. However, the invention is not limited thereto. That is, the invention is not limited to HbA1c and glico albumin as long as an index indicates a diabetes state. Another index such as the value of 1,5-anhydroglucitol (1,5-AG) or urinary sugar may be adopted. Further, the index indicating the physical condition of the user is not limited to the index indicating the diabetes state, but an index (for example, weight, a waist circumferential diameter, and BMI) indicating a state of a lifestyle habit disease such as obesity may be used or another index may be used.

In each of the foregoing embodiments, on the displayed integrated value presentation screen ESB, the integrated value has been presented by presenting the graph indicating the change in the integrated value in the predetermined period. However, the invention is not limited thereto. For example, the integrated value up to that day may be included in the result presentation screen ESA. That is, the integrated value may not be indicated by the graph and only the integrated value may be configured to be presented. On the other hand, only the evaluation based on the integrated value may be configured to be presented. That is, at least one of the sum value of the points of that day and the evaluation of the physical condition according to the sum value may be configured to be presented on the result presentation screen ESA, and at least one of the integrated value and the evaluation may be configured to be presented to the user on the integrated value presentation screen ESB. Further, at least one of the screens ESA and ESB may be configured to be presented.

The graph indicating the change in the integrated value and the graph indicating the change in the blood-sugar level have been presented on the integrated value presentation screen ESB. However, the invention is not limited thereto. For example, the graph indicating the change in the blood-sugar level may not be provided.

In each of the foregoing embodiments, to match the determination period of the average blood-sugar level determined by the value of HbA1c or the value of glico albumin, the period (the predetermined period) in which the sum value of each day is integrated to calculate the integrated value has been set to the period of the previous 90 days from the current date or the period equal to or greater than one previous week and equal to or less than two previous weeks from the current date. However, the invention is not limited thereto. For example, a predetermined period may be set to 30 days and the predetermined period may be appropriately changed to be a period less than one previous week or a period exceeding 90 days.

In each of the foregoing embodiments, the calculation rule has been adopted in which + points are added for a behavior of a direction for improving a physical condition such as diabetes and − points are added for a behavior of a direction for degrading the physical condition. However, the invention is not limited thereto. For example, a calculation rule may be adopted in which − points are added in a behavior for improving a physical condition and + points are added in a behavior for degrading the physical condition, or a point subtraction type or point addition type calculation rule may be adopted. That is, the calculation rule and the items are not limited to the content described in each of the foregoing embodiments, but other calculation rules and other items may be adopted.

In each of the foregoing embodiments, “diet,” “exercise,” and “lifestyle habit” have been exemplified as the behaviors for which the points are calculated. However, the invention is not limited thereto. That is, as described above, other behaviors may be added and at least one of “diet,” “exercise,” and “lifestyle habit” may be excluded. Further, allocations of the points for each item may also be appropriately changed. Furthermore, at least one item which is classified to a daily behavior of the user and in which the calculation rule is set may be included.

In each of the foregoing embodiments, the information processing apparatus 3 has acquired the behavior information from the detection apparatus 2 having the function of the pulsimeter and has calculated the points for the exercise based on the behavior information. However, the invention is not limited thereto. That is, the detection apparatus 2 may be configured as a detection apparatus having a function of an active amount meter that measure consumed calories. In this case, the information processing apparatus 3 may calculate points for the exercise based on the consumed calories or the like. A detection apparatus and a physical condition presentation apparatus having the functions of the pulsimeter and the active amount meter may be used. The information processing apparatus may also acquire the behavior information from each of the pulsimeter and the active amount meter which are detection apparatuses. On the other hand, as described above, the detection apparatus is not a requisite constituent of the physical condition presentation system according to the invention.

The initial setting information and the target information have been input by the user and the calculation information has been stored in the storage unit in advance or acquired from the server SV. However, the invention is not limited thereto. For example, the initial setting information, the target information, and the calculation information may be acquired from an external apparatus, a server, or the like connected to the detection apparatus, the information processing apparatus, or the physical condition presentation apparatus. The calculation information may be input the user.

In each of the foregoing embodiments, the points according to the exercise distance at the time of the aerobic exercise have been calculated and the points have been added to the daily sum value. However, the invention is not limited thereto. That is, the points according to the exercise distance may not be added. In this case, the detection apparatus and the detection unit may not acquire the positional information. On the other hand, the exercise distance is not limited to the exercise distance at the time of the aerobic exercise, but may be an exercise distance of one day.

In the foregoing fourth embodiment, the external apparatus 5 has communicated with the server SV as well as the physical condition presentation apparatus 4 and has transmitted the behavior information and the presented information received from the physical condition presentation apparatus 4 to the server SV to register the behavior information and the presented information. However, the invention is not limited thereto. That is, the server SV may be excluded. One of the behavior information and the presented information may be set as the information registered in the server SV. Instead of or in addition to the behavior information and the presented information, other information may be transmitted to the server SV via the external apparatus 5 by the physical condition presentation apparatus 4. Further, the physical condition presentation apparatus 4 may directly transmit the information to the server SV.

In each of the foregoing embodiments, the information processing apparatuses 3 and 3B and the physical condition presentation apparatus 4 have stored the physical condition presentation application in the storage units 35 and 26, and the control units 37, 38, and 29 have executed the physical condition presentation application to function as the physical condition presentation apparatus. However, the invention is not limited thereto. That is, the apparatus may be configured as an apparatus having only the function of the physical condition presentation apparatus.

The physical condition presentation application is not limited to the configuration in which the physical condition presentation application is stored in the storage unit. For example, the physical condition presentation application may be recorded on a recording medium such as an optical disc. At the time of execution of the physical condition presentation application, the physical condition presentation application may be configured to be read from the recording medium to be executed, or the physical condition presentation application may be configured to be delivered from a server or the like via a network.

In the foregoing embodiments, the configurations and the layouts illustrated in FIGS. 7 to 19 have been described for the execution screens ES (ES1 to ES9 and ESA to ESD). However, the invention is not limited thereto. That is, the execution screens ES illustrated in FIGS. 7 to 19 are merely examples and the configurations and the layouts can be appropriately modified.

What is claimed is:

1. A physical condition presentation apparatus configured:
to calculate a point corresponding to an item based on a calculation rule set for the item classified according to a behavior executed by a user and related to an index indicating a physical condition of the user and the behavior executed within a predetermined period by the user; and
to present at least one of the point and evaluation of the physical condition according to the point.

2. The physical condition presentation apparatus according to claim 1,
wherein the predetermined period is a period longer than one day, and
wherein the point is calculated per day and is integrated during the predetermined period.

3. The physical condition presentation apparatus according to claim 1, wherein the index indicating the physical condition is an index indicating a health state related to a lifestyle habit disease.

4. The physical condition presentation apparatus according to claim 3,
wherein the health state related to the lifestyle habit disease is a diabetes state, and
wherein an index indicating the diabetes state is one of hemoglobin A1c and glico albumin in blood.

5. The physical condition presentation apparatus according to claim 4, wherein when the index indicating the diabetes state is the hemoglobin A1c, the predetermined period is a period equal to or greater than past 60 days and equal to or less than past 90 days from a current date.

6. The physical condition presentation apparatus according to claim 4, wherein when the index indicating the diabetes state is the glico albumin, the predetermined period is a period equal to or greater than past 1 week and equal to or less than past 2 weeks from a current date.

7. The physical condition presentation apparatus according to claim 3, wherein the behavior includes at least diet, exercise, and life habit.

8. The physical condition presentation apparatus according to claim 1,
wherein the behavior includes exercise, and
wherein behavior information regarding the behavior of the user detected by a detection apparatus is acquired and the point in regard to exercise is calculated based on the behavior of the user indicated by the acquired behavior information.

9. The physical condition presentation apparatus according to claim 8, wherein the detection apparatus is at least one of a pulsimeter detecting a pulse rate of the user based on a pulse wave and an active amount meter calculating consumed calories of the user.

10. The physical condition presentation apparatus according to claim 1, wherein the calculated point is corrected according to a stress state of the user.

11. The physical condition presentation apparatus according to claim 1, wherein the point is calculated based on an exercise distance of the user.

12. A physical condition presentation system comprising:
a detection unit that detects behavior information regarding a behavior of a user;
a calculation unit that calculates a point corresponding to an item based on the behavior information and a calculation rule set for the item classified according to the behavior executed by the user and related to an index indicating a physical condition of the user; and
a presentation unit that presents at least one of the calculated point and evaluation of the physical condition according to the point.

13. The physical condition presentation system according to claim 12, further comprising:
a first apparatus that includes the detection unit; and
a second apparatus that includes the calculation unit and the presentation unit,
wherein the first apparatus transmits the behavior information detected by the detection unit to the second apparatus.

14. The physical condition presentation system according to claim 12, further comprising:
a first apparatus that includes the detection unit and the presentation unit; and
a second apparatus that includes the calculation unit,
wherein the first apparatus transmits the behavior information detected by the detection unit to the second apparatus,
wherein the second apparatus transmits information based on the received behavior information to the first apparatus, and
wherein the first apparatus presents the received information.

15. The physical condition presentation system according to claim 12, further comprising:
a first apparatus that includes the detection unit and the calculation unit; and
a second apparatus that includes the presentation unit,
wherein the first apparatus transmits information including at least one of the point calculated based on the detected behavior information and the evaluation of the physical condition according to the point to the second apparatus, and
wherein the second apparatus presents the received information.

16. The physical condition presentation system according to claim 12, wherein the detection unit includes a first apparatus that includes the calculation unit and the presentation unit and a second apparatus that assists the first apparatus.

17. A presented information output method executed using a presented information output apparatus that outputs information regarding a physical condition of a user, the method comprising:
calculating a point corresponding to an item based on a calculation rule set for the item classified according to a behavior executed by the user and related to an index indicating the physical condition of the user and the behavior executed within a predetermined period by the user; and
outputting at least one of the point and evaluation of the physical condition according to the point.

18. A presented information output program executed by a presented information output apparatus that outputs information regarding a physical condition of a user, the program causing the presented information output apparatus:
to calculate a point corresponding to an item based on a calculation rule set for the item classified according to a behavior executed by the user and related to an index indicating the physical condition of the user and the behavior executed within a predetermined period by the user; and
to output at least one of the point and evaluation of the physical condition according to the point.
